SEQUENCE LISTING (155 sequences)

SEQ ID NO: 1 - DNA, 2226 bp, Solanum lycopersicum
aatatactcaatctaatgaacctaattcccaaatgagtatggtattgaggcttgagtcctcatgtgtgaacttggcggtacttattaacgatcatagtacttgttgttgctacatgttgagtaatgtagttgatttcatattattacttgatatatattgctttctattttgagttggccgatgatcgtgttttgtactgacccctacttgtatgtttctttccttgttatttgtggagtgcagcaaacgtgccgtcgtctttaactcaaccgcaactctagccgatcttcattacaccggatttcagggtgagctaacgcttctagcttggactggatcttcttcttcatgtctcgatgccttgaagttccggcatgaactagcttttatttattctagctttctagatactcttagctttagtaatttgaggatagatgttcttatgatgatgacttccagattttggggataataatagttgttgagtttttagaagttatttaattgattttcattaatgaggttaagtcttccgcattatattccgtcattatattgaaatgttgggtttagattggttggttcgctcacataggaagataaatgtgggtgccactcgcggtccgttttgggtcgtgacaggtaaattagggtatcttgtggccatataaatattctccctttctttttctttaatcttatgagcgtacgataagttagtataattctaaatcctacctattaatcatcatcaattttattaaataagaaagaaaatactttttgccacctaatgtattttttattacatagaaacccgtataaaaaccccttcacacttatcttcaaactcacacacaatactcactcactagtttcatattcatattttttgaaacatgtctggtgtttggaaaatcaagaatggagtagtgaggctagttgagaacctcggtgactttcacggtgcgacgggtcgtcgtaaagtgcttgtgcacctttctagtaatgaagtaataacatcatatgcagtacttgaaaggaaactgtactctcttggatgggagaggtactatgatgaccctgaccttcttcagtaccataaaagatcaactgttcatcttatttctctaccaaacgacttcaacaacctcaggtccatgcacatgtatgatattgttgttaagaatcgtaatgagtttgctgttagggatatgtagtattactaataatcattagttgatttgagatttttctcaaattaattaatgttgtttaatttaaattaggttgtttcttcttttaacttaaggtttggtttgtgtaatttaggtcaaaggggggtgttttagtttcttttgggtgaggaagctaattattacttgttgtaatggtgtgtaagagtgaagtttatggcaataaaacttggtttcgcttcgaaacttttatctatatacttaaataaatttgtactatcaaatacttaaatttttagtcatatatatatttaaaagtcttctttatttacttaaattttgtatcaagtcaaaccagattatatttttatcattaagccaacgatgataggtggatatgtgattgatatatttttttttatggaaatatcttttcttttctctttttttttttggtcttattttgaataaagacaaaatggtattttcccatttatttcatcaagaagtctttgactataaattcaaaggctttacctcaaattcgaattcttcactgttttaaaaaaataaagtaagatgtcaagaatatatatatatatatatatatatatatatatatatatatatatatatatatatatatatatatatatatatatatatatcttttgggaaatttaattaaattattatgaagcaaataaagggtaaaagaacaaataaataaatgcaatcaaataaatgaagaggtaatatggacttgggcttttcaggctgctaatttgggttctggccctatttaaacctttgaaaacttttgtatacaacaagtgtatattgatatatacagatcgtttctaagcctttttcctgtatatcaactgtatacagcctgttctaatgcctccaacctgtatcttcatttttgtcaacatatatgttcctgaacatatagatcgctgtatacatattgtatacattatgtatacaactcatttcttgggcttttgaattatttccaat

SEQ ID NO: 2 - DNA, 321 bp, Solanum lycopersicum
atgtctggtgtttggaaaatcaagaatggagtagtgaggctagttgagaacctcggtgactttcacggtgcgacgggtcgtcgtaaagtgcttgtgcacctttctagtaatgaagtaataacatcatatgcagtacttgaaaggaaactgtactctcttggatgggagaggtactatgatgaccctgaccttcttcagtaccataaaagatcaactgttcatcttatttctctaccaaacgacttcaacaacctcaggtccatgcacatgtatgatattgttgttaagaatcgtaatgagtttgctgttagggatatgtag

SEQ ID NO: 3 - protein, 106 aa, Solanum lycopersicum
MSGVWKIKNGVVRLVENLGDFHGATGRRKVLVHLSSNEVITSYAVLERKLYSLGWERYYDDPDLLQYHKRSTVHLISLPNDFNNLRSMHMYDIVVKNRNEFAVRDM

SEQ ID NO: 4 - DNA, 75 bp, Artificial Sequence
  tracrRNA sequence
gtttagagctagaaatagcaagttaaaataaggctagtccgttatcaacttgaaaaagtggcaccgagtcggtgc

SEQ ID NO: 5 - DNA, 20 bp, Artificial Sequence
  gRNA sequence
cggtgacttccacggtgcga

SEQ ID NO: 6 - DNA, 2291 bp, Solanum lycopersicum
tttctctgtcttgtcttgaaaaaagaatgttttttttttttttataattctttactttcaattcttttacatgtgatctttagaagacaagattaaataacattttgatactttctatatattttaattataaaatcacaagattcagaagtcttgtttattttttaaaacttcatgtcaaactaaaactagataaacaaattggaacagacactatcccattgaaattttcctattgaaaaatgtccagtggctatactcacactaatgtttaaattacacaacaaaattaaaaaaaaaactcttggtattttagtgagaatttgtttctcaccatacgtttttattgacctagttaaataggaaatgggtgggaatatcacgtatcataacacaaatttctcattgatttggagtaattttttttttttaaaaaaaaattgttattagacattaattaaggattaaaagaaacatcatcaacatgagatgggacaaattaatcttccccgaaatatcttttaatttatttaattcttcctttttgtgaagggctgatcaagcaatggatataagaatagaagattgttcttagcactaaaaaaattaaagaattatgcttggaacccattaaccaaaagaattaggttcatcttatgagcataagatcattaattagtgattgtttaggagaagattctaatttcagtagggcaaattagggcatcttgtggccatttaaatattctccctttctttttctttaatcttaataaacgtacgataagttagtatatttctaaatcctataagcagccacattccaaaatcctacctattatcaattttattaaataagaaaaaagattactttttgccaccttatgtatttttttattacacactacatagaaacccctataaaaacccactcacacttatgttcaaactcacacacaatactcacttactattttcatattcatatattttttgaaacatgtctggtgtttgggtattcaagaatggagtagtgaggctagttgagaaccccggtgacttccacggtgcgacgggtcgtcgtaaagtgcttgtgcacctttctagtaatgaagtaataacatcatatgcagtacttgaaaggaaactgtactctcttggatgggagaggtactatgatgaccctgaccttcttcaattccataaaagatcaactgttcatcttatttctctaccaaaggacttcaacaacctcaagtccatgcacatgtatgatattgttgttaagaatcgtaatgagtttacagttagggatatgtagtactactaattaataattagttgatttgagatatttttctcaaattaattaatgttgtttgatttaaattaggttgtttcttgttttaacttaatgtttggtttgtgtaatttaggtttaaggggggtgttttagtttcttttgggtgaggaagctaattattacttgtaattgtgtgtaagagtgaagttttatggcaataaaaaaacttggtttggcttgaaaattttatctatatactgaaataaattcttactatcaaatacttcaattttgagtctctcacacacgcgcatatatatatatatatatatatatatatatatatatatatatatatatatacttccccgtttaaaaaagaataatcttctttcctttttagttttttttttccccgtttaaaaaagaataatcttctttcctttttagtttttttttttatataaaagaatgacttttttttggttacattttaactttagctttccacgtaattaatttagcgctacttttcaattacaaattctgctttattaaatctggttaatgatatttgaaaaattttaatttgtgaggcaaattttaggttaagatactcgaagagtttttcttaagatagttcacataaggttttgcaaaagttgggagaaattgttatatttgaactagccctatttctagcttatgtatgaatttgaaataataataatttaactatcaaattaattatgtatacaagataactcgaataatttgtatatagattatctctaacagatgccttgtagggtattaaatttgcctgcaaggctttttccagtttgttttctgtataataatatgtagcatggcatctattcccttttttaataaatatctattcataatcagacgtctaaaattcgaatacttttcttgataatatcgtcttactccttaattagtaagttgtgttgtcattaaatat

SEQ ID NO: 7 - DNA, 321 bp, Solanum lycopersicum
atgtctggtgtttgggtattcaagaatggagtagtgaggctagttgagaaccccggtgacttccacggtgcgacgggtcgtcgtaaagtgcttgtgcacctttctagtaatgaagtaataacatcatatgcagtacttgaaaggaaactgtactctcttggatgggagaggtactatgatgaccctgaccttcttcaattccataaaagatcaactgttcatcttatttctctaccaaaggacttcaacaacctcaagtccatgcacatgtatgatattgttgttaagaatcgtaatgagtttacagttagggatatgtag

SEQ ID NO: 8 - protein, 106 aa, Solanum lycopersicum
MSGVWVFKNGVVRLVENPGDFHGATGRRKVLVHLSSNEVITSYAVLERKLYSLGWERYYDDPDLLQFHKRSTVHLISLPKDFNNLKSMHMYDIVVKNRNEFTVRDM

SEQ ID NO: 9 - DNA, 20 bp, Artificial Sequence
  gRNA sequence
tatggaattgaagaaggtca

SEQ ID NO: 10 - DNA, 20 bp, Artificial Sequence
  gRNA sequence
cggtgacttccacggtgcga

SEQ ID NO: 11 - DNA, 2857 bp, Solanum lycopersicum
tttattagagatgtcatttgataatatttttattatttcttcttcttattattttttggttaagttatcttcttttcttttttttctctctttctatatttttaccatttaacgaaaataaataaataaattacttttatattttcaaaatgacatagttgaaccttatcaaggtgtttaaaatataaaaagtctacttgaaatgtttaaaagtgaaagtttatgttacttttaaggatttgacgatgaattttagtatcctaccatatatttgaaacagcttgtctcatcattgtggtacaaatgataagataaatattttttttttgttttttgtttttcatccggtgttcgatatcaacaatggaacccaataatattcagattcttacgaaacgtcctacatctgagggtaaaatactccttaacagagatgactccatagttagagaggataaataatctcaagatcactaaattaatatccctaaccaaatacaagataaaatgtgtcccacaattataactccctatatcccactttatacgacacttttcagatttcgacattcaaacaattctattttttaccgtaaaaaatatcatatcttgaattatcaatacaaatatataatttcatttaatttttaaaaaagattccattagtaaattttcaattaagcttaaactaaacagaaaaaaaatatctcttatccatcgtaccaaacgacaccagaacataaaaattaaaaaacctagaaagtaaatgaactagtatcccaaaaaggttaatagtagtccagtcattcaaaagatcagtgatcacatgatgtactagcaaacctacatacacagtggaatatatctactgctccataagaaattatttcatcatttctctaagagttatgaattattttattattatttttctttctccatctccatatattgttggagttggaaactaatataaagtaaattaaaccattatattataatgtctggcgtatggatatttgacaagaaaggtgttgcccatttgatcaaaaatcctactcgtgaatccttcgagctaaaacaaccaacacatccaggtatatgtcactcgatataagtttaactcattcgattcagtaattaatactacacattctcctttgacgatatatggagactacactatatattatatgtgttttatgtttttatgtatttgttaggggttaattagtccgttacatgctgattgcagtaatagagttaggtttttcattaagaaaaatcaaaattaaaaaatatgtaaatatagaaaaaaaatcaaggtgattcaaaaaggagttgtaatctcacgtatatatagtgaaatttatttctaaggaggtttgaatatcgaaacctagttgcacccataattacaacctttaattttggatcgtgacagatatgatattagaacatagtttattggtttcaacaaggaattgacaacataagctttaagtcaaggcaaaatgtatatattatcttccttcatgacattttgtactcgtactgactctaaattctgtattcgtccttgtaggcacgggtacagccacagcaccgggggcacgccctcgagtgttggtgtacctaccagagaatgagatgataggttcctatgaagaactagagaagagactcattgaaatcgggtggacccgattcaacaacccgatgaagtcggatcttctgcagtttcataaatcagatgattctgcacatctcatttcacttccaaagagctttacaaacttcaactcacacaatatgtatgacattgtggtcaagaatccatcggtttttgaagttcgtgatgttaaagtgtgtgatcatcttatatgaaacttaaattattaaagtaaacatacgtaattatgttatttctcctctttcgtatctgagcttgattctttttcattagccaaatacgtggatttatatgttttttcgctctttggttggtggagagatttggaacctagctagcatatctcgtgcctattctgataacatattgaattgtatacatgatcgtttcatctaaaaattaagcatttaaaaatcacatttttaattacttaactaattatatagttatattatgtgttaacacataccttgcatatatgagtttgattcttcttcatgaccagaaaatgtcaaatatatttttgctttttgagccggaactcttacatgctccatttgataacaagttggaatgtgtggttatcttatataaaaacaacacaagttgttatgaaaagcatatttataattattaatctaattatattatgttttaacacagttttttatatgtgaatttgattctttttcattgggcaaacatgtgtgaaacaatttatttttttatagactaggatgatagagaaatttgaacttaaaatctctcatgtgttcgaataacatattataaagtatgctatcattcatttaaaatttaacttattagaaaataatacacttttttttacttaattataatgtgactcttcgttttggccataataaaagtctatttgaattgatttttgacttttgattttcaagtcaatgtttgaattatttttgatgtttttagcttaaagcaaatggtttgtgcgtccaaaaaatatttgaaactattttaacttaaaatcacttaaaacaagtcgatccatgtaacatgcaagttttgacctaattagaaggtttgaaattatacctagctagagctatctatttcttttattatcaatttttttaatatatcatagttctatattaatatttttttgctttctcgata

SEQ ID NO: 12 - DNA, 887 bp, Solanum lycopersicum
atgtctggcgtatggatatttgacaagaaaggtgttgcccatttgatcaaaaatcctactcgtgaatccttcgagctaaaacaaccaacacatccaggtatatgtcactcgatataagtttaactcattcgattcagtaattaatactacacattctcctttgacgatatatggagactacactatatattatatgtgttttatgtttttatgtatttgttaggggttaattagtccgttacatgctgattgcagtaatagagttaggtttttcattaagaaaaatcaaaattaaaaaatatgtaaatatagaaaaaaaatcaaggtgattcaaaaaggagttgtaatctcacgtatatatagtgaaatttatttctaaggaggtttgaatatcgaaacctagttgcacccataattacaacctttaattttggatcgtgacagatatgatattagaacatagtttattggtttcaacaaggaattgacaacataagctttaagtcaaggcaaaatgtatatattatcttccttcatgacattttgtactcgtactgactctaaattctgtattcgtccttgtaggcacgggtacagccacagcaccgggggcacgccctcgagtgttggtgtacctaccagagaatgagatgataggttcctatgaagaactagagaagagactcattgaaatcgggtggacccgattcaacaacccgatgaagtcggatcttctgcagtttcataaatcagatgattctgcacatctcatttcacttccaaagagctttacaaacttcaactcacacaatatgtatgacattgtggtcaagaatccatcggtttttgaagttcgtgatgttaaagtgtgtgatcatcttatatga

SEQ ID NO: 13 - protein, 132 aa, Solanum lycopersicum
MSGVWIFDKKGVAHLIKNPTRESFELKQPTHPGTGTATAPGARPRVLVYLPENEMIGSYEELEKRLIEIGWTRFNNPMKSDLLQFHKSDDSAHLISLPKSFTNFNSHNMYDIVVKNPSVFEVRDVKVCDHLI

SEQ ID NO: 14 - DNA, 20 bp, Artificial Sequence
  gRNA sequence
agagactcattgaaatcggg

SEQ ID NO: 15 - DNA, 20 bp, Artificial Sequence
  gRNA sequence
agaagagactcattgaaatc

SEQ ID NO: 16 - DNA, 2227 bp, Solanum lycopersicum
aagttttgaattctttaggttgctttttctttaatttttttcttcttctcatatcatgaatcttatccatttcaatatttccaccaaacatgggacatggacatctctatgagttcatcttcttgcttccaatgcattatctggtgtttgatattcgtattgagcttccactaattcagattcatgccgcataaagtctatttaaaagaaaaatatttctatcaaaattgttttcatactctagggtcgagcaaagggattcatgaccaatgatatctacgggaatattaaagaatcttgataaagaacacttctccttgtccgagcctttgacaaaaatcatttttggtaggattgcttccccacctttcagtcttatgtagaatttgaattagttgagattcactatgaatatcgaataaataacaaaaaaaaaaaggagtaatgaatctttccaaatatagaatatattatgattaaatgcatgcatgggaagcaaaaagatgaacttatggagatgtgtcatgtcccatatatttgatggaaatattgggttggataagattcatgatgaaaaaaaaaagcggtgacataaatctgaattagtcggaactccaaatagtttaatttgtttttgaaaaataaccttcttttacttgccctttccttttttatctcttcaaaaaataaaaataaaacttcttaccacaatttatactatatattacttattaaggggaatcttgatgcaataacataacacagttatctttatcagattcgaaccgtagaagcagctacaaatatttgtaataaggaaggctatttacatcacacatgtatttatacgtatatggacttatttatttatttatatatatatatatatatatatgcatatcacaccatgcattaaccctataaaacccacacattatattctttttcaacaacaccatcttttacatatattcaacttcccctccctctatccctcatcatgtcaggtgtttggattttcaaaaacggcgtcgtccggctagaaacccccggtgactgccacgtcagctccacgaccggtcatcggaaagttctagtacatgttcctagtaaagaagtcattacatgttatgcaaatcttgaaaaaaagctttatagtcttggatgggaaaggtattatgatgatccacaacttcttcaataccacaaaagatccacaattcatcttatttccctcccaattgattttaataggtttaaatccattcatatgtatgatattgttgttaaaaatcgaaatgaatttgaagttagagatatgtaaagttactaactttctttacgtggatataagaaatgtgaaatttggagaaacttatgtgttttcgagttgatagtgatatgtttggagattggagttgtgtttgaacatggatacgaacggaattgtttttgaatttttgaaagtgaaaattgctttttattgtttttgaacttaaaattgttatgtggctaacaaaataaaatcaatcaacaaacaagtcgttgtagtatagtggtaagtattcccgcctgtcacgcgggtgacccgggttcgatccccggcaacggcgttaattttttttatgtttctacacataccatatatctagttatatcttacgacaagcacaaatacattatgctctcgcaacatacaatgtatctagttatatatcttacgagaagcacaaatacattatgctctcgcaacatacaatatatccagttgtgtcttacgacaagtactccaaaacccaccaacgctcgagaaatgccttgttatggtgtaagaaacatcagcttcagtatgttaagactgataacaaaggagttacttcacaagttctttttcaacaagtaatttacatagagtttggatgttgtgttctggacaacaagaaaaaatgaatgtagttagtctaaggctatgttgcttggactctccaaaagatgctacacccgtgtcgggtcctccaaaaatgcactacttttgaaggatcagacatgcacgtgtcgccatatttcaagagcccgagcaacataggttcaaggaactcatatgatataggctaatgtcacgaactcactttcttctttgtcgtgctccaaatgtttcagctctgaacctatacattccgccatccaatatatctcctcagtccgcgggtgagacttgtcatccgat

SEQ ID NO: 17 - DNA, 324 bp, Solanum lycopersicum
atgtcaggtgtttggattttcaaaaacggcgtcgtccggctagaaacccccggtgactgccacgtcagctccacgaccggtcatcggaaagttctagtacatgttcctagtaaagaagtcattacatgttatgcaaatcttgaaaaaaagctttatagtcttggatgggaaaggtattatgatgatccacaacttcttcaataccacaaaagatccacaattcatcttatttccctcccaattgattttaataggtttaaatccattcatatgtatgatattgttgttaaaaatcgaaatgaatttgaagttagagatatgtaa

SEQ ID NO: 18 - protein, 107 aa, Solanum lycopersicum
MSGVWIFKNGVVRLETPGDCHVSSTTGHRKVLVHVPSKEVITCYANLEKKLYSLGWERYYDDPQLLQYHKRSTIHLISLPIDFNRFKSIHMYDIVVKNRNEFEVRDM

SEQ ID NO: 19 - DNA, 20 bp, Artificial Sequence
  gRNA sequence
ccctcatcatgtcaggtgtt

SEQ ID NO: 20 - DNA, 20 bp, Artificial Sequence
  gRNA sequence
ttttcaaaaacggcgtcgtc

SEQ ID NO: 21 - DNA, 315 bp, Solanum lycopersicum
atgtctggagtttgggtattcaagaatggtgttgtccgtctagtggagaactccgattgccacggggcgaacggactccgaaaagttcttgtacatcttcctagtaatgaagtcatcacatcatatgcagtacttgaaaggaaactgtactctcttggatgggagaggtactatgatgaacctgaacttcttcaataccacaaaagatcaaccgttcatcttatttctctaccaaaggatttcaacaggttcaaatccatgcatatgttcgatatcgtcgtcaagaatcgcaatgaatttgaggttagagatatg

SEQ ID NO: 22 - protein, 105 aa, Solanum lycopersicum
MSGVWVFKNGVVRLVENSDCHGANGLRKVLVHLPSNEVITSYAVLERKLYSLGWERYYDEPELLQYHKRSTVHLISLPKDFNRFKSMHMFDIVVKNRNEFEVRDM

SEQ ID NO: 23 - DNA, 26 bp, Solanum lycopersicum
ttttatggaattgaagaaggtcaggg

SEQ ID NO: 24 - DNA, 22 bp, Artificial Sequence
  disrupted locus sequence
ttttatggaattgaggtcaggg

SEQ ID NO: 25 - DNA, 27 bp, Solanum lycopersicum
ccccggtgacttccacggtgcgacggg

SEQ ID NO: 26 - DNA, 22 bp, Artificial Sequence
  disrupted locus sequence
ccccggtccacggtgcgacggg

SEQ ID NO: 27 - DNA, 27 bp, Solanum lycopersicum
cctcggtgactttcacggtgcgacggg

SEQ ID NO: 28 - DNA, 22 bp, Artificial Sequence
  disrupted locus sequence
ccccggttcacggtgcgacggg

SEQ ID NO: 29 - protein, 108 aa, Nicotiana benthamiana
MSGVWIFDKKGVARLITNPTRESFDLMQPTSSGTGTAPGARPKVLVYLPENQVISSYADLEKILIELGWSRYNNPIRLDFMQFHKSDDSAHLISLPKEFTNFKSLHIE

SEQ ID NO: 30 - protein, 130 aa, Petunia axillaris
MSGVWIFDKKGVAHLIKNPTRESFELKEPTYPGTGTATAPGARPKVLVYLPENEVISSYDELEKILVELGWSRYNNPTRSDLLQFHKSDDSAHLISLPISFTNFKPLHMYDIVVKNRSFFEVRDSPYTSY

SEQ ID NO: 31 - protein, 130 aa, Petunia inflata
MSGVWIFDKKGVAHLIKNPTRESFELNEPTYPGTGTATAPGARPKALVYLPENEVISSYDELEKILIELGWSRYNSPTRSDLLQFHKSNDSGHLISLPISFTNFKPLHMYDIVVKNRSFFEVRDSPYTSY

SEQ ID NO: 32 - protein, 132 aa, Capsicum annuum Zunla
MSGVWIFDKKGVAHLIKNPTRESFELKQPAYPGTGTATAPGARPRVLVYLPENEMISSYEELERRLIELGWTRFNNPMRSDLLQFHKSDDSAHLISLPKSFTDFKSLHMYDIVVKNPSFFEVRNAEVDNHLI

SEQ ID NO: 33 - protein, 132 aa, Capsicum annuum Zunla
MSGVWIFDKKGVAHLIKNPTRESFELKQPAYPGTGTATAPGARPRVLVYLPENEMISSYEELERRLIELGWTRFNNPMRSDLLQFHKSDDSAHLISLPKSFTNFKSLHMYDIVVKNPSFFEVRNAEVDNHLI

SEQ ID NO: 34 - protein, 100 aa, Capsicum annuum
GTGTATAPGARPRVLVYLPENEMISSYEELERRLIELGWTRFNNPMRSDLLQFHKSDDSAHLISLPKSFTNFKSLHMYDIVVKNPSFFEVRNAEVDNHLI

SEQ ID NO: 35 - protein, 132 aa, Solanum melongena
MSGVWIFDKKGVAHLIKNPTRESFELKQSTYPGTGTATAPGARPRVLVYLPENEIISSYEELERRLIELGWTRFNNPMKSDLLQFHKSDDSAHLISLPKSFTNFKSLQMYDIVVKNPSFFEVRDIKVYDHPI

SEQ ID NO: 36 - protein, 132 aa, Solanum tuberosum
MSGVWIFDKKGVAHLIKNPTRESFELKQPTYPGTGTVTAPGARPRVLVYLPENEMISSYEELEKRLIELGWTRFNNPMKSDLLQFHKSDDSAHLISLPKSFTNFKSHQMYDIVVKNPSIFEVRDVKVCDHLI

SEQ ID NO: 37 - protein, 132 aa, Solanum pennellii; identical to SEQ ID NO: 13

SEQ ID NO: 38 - protein, 123 aa, Solanum lycopersicum
MSGVWIFDKKGVAHLIKNPTRESFELKQPTHPGTGTATAPGARPRVLVYLPENEMIGSYEELEKRLIEIGWTRFNNPMKSDLLQFHKSDDSAHLISLPKSFTNFNSHNMYDIVVKNPSVFEVR

SEQ ID NO: 39 - protein, 111 aa, Nicotiana benthamiana
MSGVWLSKNTGVIRLLENQTEEEQSIGRKRKVLVHLPTQEIVSSYNSLDKILTDLGWEKYDCGDDPHFYQFHKRTPIHLSLSLPNDFAKFNTVQMYDIVFKTRHIFHVRYI

SEQ ID NO: 40 - protein, 72 aa, Petunia inflata
MSGVWVFKNGVFRLVENPGAEQAQRRRKVLVHLPTGQMVSSYCSLERILNGLGWERVQEIVLKYCVGIKLSH

SEQ ID NO: 41 - protein, 106 aa, Petunia axillaris
MSGVWVFKNGVFRLVENPGAEQAQRRRKVLVHLPTGQMVSSYCSLERILNGLGWERYYGGDPELFQFHKHSSIDLISLPKDFSKFNSIHMYDIVVKNPNVFHVRDA

SEQ ID NO: 42 - protein, 106 aa, Solanum melongena
MSGVWVFKNGVFRLVENGSGDQAQRRRKVLIHLPSGQVVSSYCSLERILNDLGWERYYEGDAELFQFHKHSSIDLISLPMDFTKFNSIHMYDIVVKNPNIFHVRDV

SEQ ID NO: 43 - protein, 106 aa, Capsicum annuum Zunla
MSGVWVFKNGVFRLVENGSGDQAQRRRKVLLHLPSGQVVSSYCSLERILNGLGWERYYGGDTELFQFHKHSSIDLISLPKDFAKFNSIHMYDIVVKNPNVFHVRDV

SEQ ID NO: 44 - protein, 122 aa, Capsicum annuum
LIFEKEHTHTHTSEVEMSGVWVFKNGVFRLVENGSGDQAQRRRKVLLHLPSGQVVSSYCSLERILNGLGWERYYGGDTELFQFHKHSSIDLISLPKDFAKFNSIHMYDIVVKNPNVFHVRDV

SEQ ID NO: 45 - protein, 106 aa, Nicotiana benthamiana
MSGVWVFKNGVFRLVENPSSEQGQRRRKVLVHLPTGQVVSSYCSLERILKGLGWERYYGGDPELFQFHKHSSIDLISLPKEFAKFNSIHMYDIVVKNPNVFHVRDA

SEQ ID NO: 46 - protein, 106 aa, Nicotiana benthamiana
MSGVWVFKNGVFRLVENPSSEQGQRRRKVLLHLPTGQVVSSYCSLERILNGLGWERYYGGDPELFQFHKHSSIDLISLPKDFAKFNSIHMYDIVVKNPNVFHVRDV

SEQ ID NO: 47 - protein, 113 aa, Solanum pennellii
MPGVWEIKNGVVRLVEKPGDSHGATVRNKVLVHLSSNEVITSYASLERILISIGWERYYDGDPDLLQYHKRSTVHIISLPKDFKNFKFPHMLDIVLKNRNDFTTRDTSITNNN

SEQ ID NO: 48 - protein, 104 aa, Nicotiana benthamiana
MSGVWVFKNGVLRLVENPGDHFQGCRKVLVHIPTNEVITSYAILETKLYNLGWERYYDDPELLQYHKRCTTHLISLPKDFNKFKTMHMYDIVVKNRNEFEVRDM

SEQ ID NO: 49 - protein, 106 aa, Solanum melongena
MSGVWVFKNGVVRLVENTGDCQGANGGRKVLVHVPSDEVITSYAVLERKLYSLGWERYYDDPDLLQYHKRSTVHLISLPKDFNRFKSMHMYDIVVKNRNEFEVREM

SEQ ID NO: 50 - protein, 105 aa, Capsicum annuum Zunla
MSGVWVFKNGVVRLVENGDCQGVNGCRKVLVHLASGEVITSYAVLERKLYSLGWERYYDEPELLQYHKRSTVHLISLPKDFNRFKSMHMFDIVVKNRNEFEVRDM

SEQ ID NO: 51 - protein, 133 aa, Solanum lycopersicum
SHHKLQTHTKNILIHLSSTKNITTSSTIMSGVWVFKNGVVRLVENSDCHGANGLRKVLVHLPSNEVITSYAVLERKLYSLGWERYYDEPELLQYHKRSTVHLISLPKDFNRFKSMHMFDIVVKNRNEFEVRDM

SEQ ID NO: 52 - protein, 105 aa, Solanum pennellii
MSGVWVFKNGVVRLVENSDCHGANGLRKVLVHLPSNEVITSYAVLERKLYSLGWERYYDEPELLQYHKKSTVHLISLPKDFNRFKSMHMFDIVVKNRNEFEVRDM

SEQ ID NO: 53 - protein, 105 aa, Solanum pimpinellifolium; identical to SEQ ID NO: 22

SEQ ID NO: 54 - protein, 105 aa, Solanum tuberosum
MSGVWVFKNGVVRLVENSDCHGANGLRKVLVHLPSDEVITSYAVLERKLYSLGWERYYDEPELLQYHKRSTVHLISLPKDFNRFKSMHMFDIVVKNRNEFEVRDM

SEQ ID NO: 55 - protein, 102 aa, Nicotiana benthamiana
MSGVWVFKNGVVRLEDCQGSSGRRKVLVHVPSNEVITSYPVLERKLYSLGWERYYDDLNLLQYHKRSTVHLISLPKDFNKFKSMHMYDIVVKNRNEFEVRDT

SEQ ID NO: 56 - protein, 106 aa, Petunia axillaris
MSGVWVFKNGVVRLVENPGDCQGSSGRRKVLVHVPTNEVITSYALLERKLYSLGWERYYDDPDLLQYHKRSTVHLISLPKDFNKFKSIHMYDIVVKNRNEFEVRDK

SEQ ID NO: 57 - protein, 106 aa, Petunia inflata
MSGVWVFKNGVVRLVENPGDCQGSSGRRKVLVHVPTNEVITSYALLERKLYSLGWERYYDDPDLLQYHKRSTVHLISLPKDFNKFKSIQMYDIVVKNRNEFEVRDK

SEQ ID NO: 58 - protein, 198 aa, Petunia axillaris
MSGVWVFKNGVLRLVENPGDNYHGSRKVLVHVPSDEVITSYAILERKLYNLGWERYYDDPNLLQYHKRSTVHLISLPRDFSKLKTMHMYDIVVKNRNEFESNGVVRLENPSDYHGSAGRRKVLVHAASNEVITSYATLERKLYNLGWERYYDDPELLQYHKRSTVHLISLPKDFSRFKSMHMYDIVVKNRNEFEVRDM

SEQ ID NO: 59 - protein, 197 aa, Petunia inflata
MSGVWVFKNGVLRLVENPGDNYHGSRKVLVHVPSNEVVTSYAILERKLYNLGWERYYDDPNLLQYHKRSTVHLISLPRDFSKFKTMHMYDIVVKNRNEFESNGVVRLENPGDYHGSSGRRKVLVHATSNEVITSYATLERKLYNLGWERYYDDPELLQYHKRSTVHLISLPKDFSRFKSMHMYDIVVKNRNEFEVRD

SEQ ID NO: 60 - protein, 106 aa, Capsicum annuum Zunla
MSGVWVFKNGVVRLVENPGDCHGATGRRKVLVHLASNEVISSYASLERKLYSLGWERYYDDPDLLQYHKRSTVHLISLPKDFNRFKSMHMYDIVVKNRNEFEVRDI

SEQ ID NO: 61 - protein, 106 aa, Petunia axillaris
MSGVWVFKNGVVRLVENPGDFHGSSGRRKVLVHVPSNEVISSYATLERKLSSLGWERYYDDPDLLQYHKRSTVHLISLPKDFNRFKSMHMYDIVVKNRNEFEVRDM

SEQ ID NO: 62 - protein, 106 aa, Petunia inflata
MSGVWVFKNGVVRLVENPGDFHGSTGRRKVLVHVPSNEVISSYATLERKLSSLGWERYYDDPDLLQYHKRSTVHLISLPKDFNRFKSMHMYDIVVKNRNEFEVRDM

SEQ ID NO: 63 - protein, 106 aa, Solanum melongena
MSGVWVFKNGVVRLVENPGDFHGSTGRRKVLVHLPSNEVITSYAALERKLYSLGWERYYDDPDLLQYHKRSTVHLISLPKDFNRFKSMHMYDIVVKNRNEFEVRDM

SEQ ID NO: 64 - protein, 106 aa, Nicotiana benthamiana
MSGVWVFKNGVVRLVENPGDCHGATGRRKVLVHLSSNEVITSYSALERKLYSLGWERYYDDPDLLQYHKRSTVHLISLPKDFNKFKSMHMYDIVVKNRNEFEVRDM

SEQ ID NO: 65 - protein, 106 aa, Nicotiana benthamiana
MSGVWVFKNGVVRLVENPGDCHGATGRRKVLVHLSSNEVITSYSALERKLYSLGWERYYDDPDLLQYHKRSTVHLISLPKDFNRFKSMHMYDIVVKNRNEFEVRDM

SEQ ID NO: 66 - protein, 106 aa, Solanum lycopersicum; identical to SEQ ID NO: 8

SEQ ID NO: 67 - protein, 106 aa, Solanum pennellii
MSGVWVFKNGVVRLVENPGDFHGATGRRKVLVHLSSNEVITSYASLERILFSLGWERYYDDPDLLQFHKRSTIHLISLPKDFNNFKSMHMYDIVVKNRNEFTVRDM

SEQ ID NO: 68 - protein, 106 aa, Solanum tuberosum
MSGVWVFKNGVVRLVENAGDFHGATGRRKVLVHLSSNEVITSYASLERNLYSLGWERYYDDPDLLQFHKRSTVHLISLPKDFNRFKSMHMYDIVVKNRNEFEVRDM

SEQ ID NO: 69 - protein, 106 aa, Solanum pennellii
MSGVWKIKNGVVRLVENLGDFQGATGRRKVLVHLSSNEVITSYASLERILYSLGWERYYDDPNLLQYHKRSTVHLISLPKDFNNLKSMHMYDIVVKNRNEFTVRDM

SEQ ID NO: 70 - protein, 106 aa, Solanum lycopersicum; identical to SEQ ID NO: 3

SEQ ID NO: 71 - protein, 108 aa, Capsicum annuum Zunla
MSGVWTFKNGVVRLENRGDCHVSATTGRRKVLVHVASDEVITCYENLERKLCNLGWERFKSMHMYDIVVKNRNEFEVRDMWATRSTALRCEVQVMMDQPEVCADALDK

SEQ ID NO: 72 - protein, 86 aa, Capsicum annuum
VSATAGRRKVLVHVASDEVITCYENLERKLCNLGWERYYDDPQLLQYHKRSTIHLISLPLDFTRFKSMHMYDIVVKNRNEFEVRDM

SEQ ID NO: 73 - protein, 107 aa, Solanum melongena
MSGVWVFKNGVVRLENPGDCHVSSTTSRRKVLVHVPSNEVITCYENLERKLYSLGWERYYDDPQLLQYHKRSTIHLISLPMDFNRFKSMHMYDIVVKNRNEFEVRDM

SEQ ID NO: 74 - protein, 107 aa, Solanum lycopersicum; identical to SEQ ID NO: 18

SEQ ID NO: 75 - protein, 107 aa, Solanum tuberosum
MSGVWIFKNGVVRLENPGDCHVSSTTGHRKVLVHVPSNEVITCYANLERKLYSLGWERYYDDPQLLQYHKRSTIHLISLPIDFNRFKSIHMYDIVVKNRNEFEVRDM

SEQ ID NO: 76 - DNA, 20 bp, Artificial Sequence
  gRNA sequence
gagaagagactcattgaaat

SEQ ID NO: 77 - DNA, 20 bp, Artificial Sequence
  gRNA sequence
gggggcacgccctcgagtgt

SEQ ID NO: 78 - DNA, 20 bp, Artificial Sequence
  gRNA sequence
ggtaggtacaccaacactcg

SEQ ID NO: 79 - DNA, 20 bp, Artificial Sequence
  gRNA sequence
aacactcgagggcgtgcccc

SEQ ID NO: 80 - DNA, 20 bp, Artificial Sequence
  gRNA sequence
gggtacagccacagcaccgg

SEQ ID NO: 81 - DNA, 20 bp, Artificial Sequence
  gRNA sequence
cgggtacagccacagcaccg

SEQ ID NO: 82 - DNA, 20 bp, Artificial Sequence
  gRNA sequence
acgggtacagccacagcacc

SEQ ID NO: 83 - DNA, 20 bp, Artificial Sequence
  gRNA sequence
cacgggtacagccacagcac

SEQ ID NO: 84 - DNA, 20 bp, Artificial Sequence
  gRNA sequence
agggcgtgcccccggtgctg

SEQ ID NO: 85 - DNA, 20 bp, Artificial Sequence
  gRNA sequence
tgtattcgtccttgtaggca

SEQ ID NO: 86 - DNA, 20 bp, Artificial Sequence
  gRNA sequence
aattctgtattcgtccttgt

SEQ ID NO: 87 - DNA, 20 bp, Artificial Sequence
  gRNA sequence
tggctgtacccgtgcctaca

SEQ ID NO: 88 - DNA, 20 bp, Artificial Sequence
  gRNA sequence
acgagtacaaaatgtcatga

SEQ ID NO: 89 - DNA, 20 bp, Artificial Sequence
  gRNA sequence
acaacataagctttaagtca

SEQ ID NO: 90 - DNA, 20 bp, Artificial Sequence
  gRNA sequence
ttgtaattatgggtgcaact

SEQ ID NO: 91 - DNA, 20 bp, Artificial Sequence
  gRNA sequence
agtaggatttttgatcaaat

SEQ ID NO: 92 - DNA, 20 bp, Artificial Sequence
  gRNA sequence
aaccattatattataatgtc

SEQ ID NO: 93 - DNA, 20 bp, Artificial Sequence
  gRNA sequence
agtcaccgggggtttctagc

SEQ ID NO: 94 - DNA, 20 bp, Artificial Sequence
  gRNA sequence
gtcgtccggctagaaacccc

SEQ ID NO: 95 - DNA, 20 bp, Artificial Sequence
  gRNA sequence
agctgacgtggcagtcaccg

SEQ ID NO: 96 - DNA, 20 bp, Artificial Sequence
  gRNA sequence
gagctgacgtggcagtcacc

SEQ ID NO: 97 - DNA, 20 bp, Artificial Sequence
  gRNA sequence
gaccggtcgtggagctgacg

SEQ ID NO: 98 - DNA, 20 bp, Artificial Sequence
  gRNA sequence
aactttccgatgaccggtcg

SEQ ID NO: 99 - DNA, 20 bp, Artificial Sequence
  gRNA sequence
gatgaattgtggatcttttg

SEQ ID NO: 100 - DNA, 20 bp, Artificial Sequence
  gRNA sequence
gagggaaataagatgaattg

SEQ ID NO: 101 - DNA, 20 bp, Artificial Sequence
  gRNA sequence
ccctcccaattgattttaat

SEQ ID NO: 102 - DNA, 632 bp, Solanum lycopersicum
catctcatcataaactacaaacacatacaaaaaacattctcattcacctttcctctacaaaaaacataacaacatcttcaacaatcatgtctggagtttgggtattcaagaatggtgttgtccgtctagtggagaactccgattgccacggggcgaacggactccgaaaagttcttgtacatcttcctagtaatgaagtcatcacatcatatgcagtacttgaaaggaaactgtactctcttggatgggagaggtactatgatgaacctgaacttcttcaataccacaaaagatcaaccgttcatcttatttctctaccaaaggatttcaacaggttcaaatccatgcatatgttcgatatcgtcgtcaagaatcgcaatgaatttgaggttagagatatgtaaacaaaatatgggggaaaaaagggaaggagttgatcatttgaatgtgtttttttttcttttttttgcttttttttggtcaagtgtgttgtaattaagtttctatcgtttaatttgtgatttgtttcacaatgttgctaaggttgtaatttggaaagttgtaagaggggaaatgttgtatattattacaagtgaatgtgttttattatatgatatatatatatataagag

SEQ ID NO: 103 - DNA, 19 bp, Artificial Sequence
  gRNA sequence
tctagtggagaactccgat

SEQ ID NO: 104 - DNA, 20 bp, Artificial Sequence
  gRNA sequence
aaaagttcttgtacatcttc

SEQ ID NO: 105 - DNA, 20 bp, Solanum lycopersicum
tcgcaccgtgaaagtcaccg

SEQ ID NO: 106 - DNA, 20 bp, Solanum lycopersicum
tcgcaccgtggaagtcaccg

SEQ ID NO: 107 - DNA, 26 bp, Solanum lycopersicum
ccgtcgcaccgtgaaagtcaccgagg

SEQ ID NO: 108 - DNA, 26 bp, Solanum lycopersicum
ccgtcgcaccgtggaagtcaccgggg

SEQ ID NO: 109 - DNA, 26 bp, Artificial Sequence
  mutant brachytic locus sequence
ccgtcgcaccgtgaaagtcaccgagg

SEQ ID NO: 110 - DNA, 27 bp, Artificial Sequence
  mutant brachytic locus sequence
ccgtcgcaaccgtggaagtcaccgggg

SEQ ID NO: 111 - DNA, 21 bp, Artificial Sequence
  mutant brachytic locus sequence
ccgtcgcaccgtgaaccgagg

SEQ ID NO: 112 - DNA, 21 bp, Artificial Sequence
  mutant brachytic locus sequence
ccgtcgcaccgtggaccgggg

SEQ ID NO: 113 - DNA, 27 bp, Artificial Sequence
  mutant brachytic locus sequence
ccgtcgcaccgtgaaagtcaaccgagg

SEQ ID NO: 114 - DNA, 21 bp, Artificial Sequence
  mutant brachytic locus sequence
ccgtcgcaccgtggaccgggg

SEQ ID NO: 115 - DNA, 19 bp, Artificial Sequence
  gRNA sequence
atcggagttctccactaga

SEQ ID NO: 116 - DNA, 20 bp, Artificial Sequence
  gRNA sequence
gaagatgtacaagaactttt

SEQ ID NO: 117 - DNA, 20 bp, Artificial Sequence
  gRNA sequence
tcgcaccgtgaaagtcaccg

SEQ ID NO: 118 - DNA, 22 bp, Artificial Sequence
  primer
ttcccctcttacaactttccaa

SEQ ID NO: 119 - DNA, 20 bp, Artificial Sequence
  primer
ccagaaacgggggagactac

SEQ ID NO: 120 - DNA, 22 bp, Artificial Sequence
  primer
catgtgcatggacttgaggttg

SEQ ID NO: 121 - DNA, 21 bp, Artificial Sequence
  primer
agggctgatcaagcaatggat

SEQ ID NO: 122 - DNA, 22 bp, Artificial Sequence
  primer
gacctgaggttgttgaagtcgt

SEQ ID NO: 123 - DNA, 21 bp, Artificial Sequence
  primer
ttttgggtcgtgacaggtaaa

SEQ ID NO: 124 - DNA, 20 bp, Artificial Sequence
  primer
ccagattcatctcggggagc

SEQ ID NO: 125 - DNA, 20 bp, Artificial Sequence
  primer
gagctgcttaaccgtgacct

SEQ ID NO: 126 - DNA, 20 bp, Artificial Sequence
  primer
ggacttcctggacaacgagg

SEQ ID NO: 127 - DNA, 20 bp, Artificial Sequence
  primer
cgtgagttcttctggccctt

SEQ ID NO: 128 - DNA, 20 bp, Artificial Sequence
  primer
gagggcgtggatatgtcctg

SEQ ID NO: 129 - DNA, 20 bp, Artificial Sequence
  primer
ggcgacctcgtattgggaat

SEQ ID NO: 130 - DNA, 20 bp, Artificial Sequence
  primer
gctctcgatgagctgatgct

SEQ ID NO: 131 - DNA, 20 bp, Artificial Sequence
  primer
atttgtgtacgcccgacagt

SEQ ID NO: 132 - DNA, 23 bp, Artificial Sequence
  off-target site
gaacgtagttgaccactagatgg

SEQ ID NO: 133 - DNA, 23 bp, Artificial Sequence
  off-target site
gattgaagttctccgttagatgg

SEQ ID NO: 134 - DNA, 23 bp, Artificial Sequence
  off-target site
caatggagttcttcactagaggg

SEQ ID NO: 135 - DNA, 23 bp, Artificial Sequence
  off-target site
gatgagagttctgcacttgatgg

SEQ ID NO: 136 - DNA, 24 bp, Artificial Sequence
  off-target site
ttatgatgtacaaaaacttttagg

SEQ ID NO: 137 - DNA, 24 bp, Artificial Sequence
  off-target site
ggaagatgtaccaatacgtttcgg

SEQ ID NO: 138 - DNA, 24 bp, Artificial Sequence
  off-target site
ttaagatttacaacaactttttgg

SEQ ID NO: 139 - DNA, 24 bp, Artificial Sequence
  off-target site
ggaagatgtcctagttctttttgg

SEQ ID NO: 140 - DNA, 24 bp, Artificial Sequence
  off-target site
ggaacatgtacaagaagcttgagg

SEQ ID NO: 141 - DNA, 24 bp, Artificial Sequence
  off-target site
ggaagacgttcaagaatttttcgg

SEQ ID NO: 142 - DNA, 24 bp, Artificial Sequence
  off-target site
ggaagatgaataataactatttgg

SEQ ID NO: 143 - DNA, 24 bp, Artificial Sequence
  off-target site
aacagaagtacaagaacttttggg

SEQ ID NO: 144 - DNA, 24 bp, Artificial Sequence
  off-target site
agcagatgtacaagatctttaagg

SEQ ID NO: 145 - DNA, 24 bp, Artificial Sequence
  off-target site
agaagctgtatatgaacttttggg

SEQ ID NO: 146 - DNA, 24 bp, Artificial Sequence
  off-target site
ggaagaagaagaagaagttttagg

SEQ ID NO: 147 - DNA, 24 bp, Artificial Sequence
  off-target site
tgatgatgtaaaagaactttttgg

SEQ ID NO: 148 - DNA, 24 bp, Artificial Sequence
  off-target site
ggaagatggacaacaagatttagg

SEQ ID NO: 149 - DNA, 24 bp, Artificial Sequence
  off-target site
tgaagaagcacaagagctttttgg

SEQ ID NO: 150 - DNA, 24 bp, Artificial Sequence
  off-target site
ggatgatatacaagcatttttagg

SEQ ID NO: 151 - DNA, 24 bp, Artificial Sequence
  off-target site
ggaagatgtaccataactttaggg

SEQ ID NO: 152 - DNA, 24 bp, Artificial Sequence
  off-target site
gcaagatgcacaagaccctttggg

SEQ ID NO: 153 - DNA, 24 bp, Artificial Sequence
  off-target site
gcaagatctacaagaacttcacgg

SEQ ID NO: 154 - DNA, 24 bp, Artificial Sequence
  off-target site
ggaagatattcaataaattttagg

SEQ ID NO: 155 - DNA, 24 bp, Artificial Sequence
  off-target site
ggaagatatgaaagaactttatgg

(19) United States
(12) Patent Application Publication
(10) Pub. No.: US 2024/0084320 A1
(43) Pub. Date: Mar. 14, 2024

(54) COMPOSITIONS AND METHODS FOR ALTERING STEM LENGTH IN SOLANACEAE

(71) Applicant: UNIVERSITY OF FLORIDA RESEARCH FOUNDATION, INCORPORATED, GAINESVILLE, FL (US)

(72) Inventor: Tong Geon LEE, LITHIA, FL (US)

(21) Appl. No.: 18/260,161

(22) PCT Filed: Jan. 5, 2022
(86) PCT No.: PCT/US2022/070033

§ 371 Date: Jun. 30, 2023

Related U.S. Application Data
(60) Provisional application No. 63/135,048, filed on Jan. 8, 2021.

(51) Int. Cl. C12N 15/82; C12N 9/22

(52) U.S. Cl. CPC C12N 15/8262; C12N 9/22; C12N 2310/20

(57) ABSTRACT

Described are CRISPR constructs and systems that can be used to generate brachytic Solanaceae plants rapidly and efficiently. Also described are methods of introducing a brachytic phenotype into a Solanaceae plant having one or more other desired traits using the described CRISPR constructs and systems to generate loss of function mutations in one or more brachytic loci in the plant.

### CROSS-REFERENCE TO RELATED APPLICATIONS

This application claims the benefit of U.S. Provisional Application No. 63/135,048, filed Jan. 8, 2021, which is incorporated herein by reference.

### REFERENCE TO A SEQUENCE LISTING SUBMITTED AS A TEXT FILE VIA EFS WEB

The Sequence Listing written in file 572399_T18366WO001_SeqListing.txt is 88 kilobytes in size, was created on Dec. 16, 2021, and is hereby incorporated by reference.

### BACKGROUND

Tomato is the most valuable horticultural crop worldwide (Food and Agriculture Organization of the United Nations). Fresh-market and processing tomatoes are the two most commonly consumed types of tomatoes and account for more than $2.6 billion in annual farm cash receipts in the United States alone (United States Department of Agriculture Economic Research Service (USDA ERS)). Unlike processing tomatoes, which have been successfully adapted for farm machinery for nearly all aspects of production, field production of fresh-market tomatoes continues to heavily rely on manual labor (Davis and Estes, 1993 USDA ERS; Van Sickle and McAvoy 2015 USDA ERS).

Most field-grown fresh-market tomato varieties have determinate vines with upright growth. Because of their heavy large fruits (typical 110-250 g for fresh-market fruits versus <80 g for processing fruits) and the higher quality requirement of exterior standards, displacement of those plants, especially fruits laying on the soil, significantly reduces yield and quality by damages from human activities, machineries and soilborne pathogens (Adelana, B. O. 1980. Relationship between lodging, morphological characters and yield of tomato cultivars. Scientia Hort. 13:143-148). Manual practices such as staking and tying are required to sustain the current production of marketable fresh-market tomatoes.

Current compact growth habit (CGH) tomato plants, while being determinate, and having shortened internodes, a spreading characteristic (with increased side branching), and a concentrated fruit setting (producing fruits over a narrow time interval) suffer from insufficient fruit size. There presently are no commercial large-fruited, fresh-market tomatoes that show CGH. Development of fresh market tomato lines that hold fruits off the ground without the support of stakes throughout a season, adapt to high plant density per the unit area, and produce high quality fresh-market fruit of economically viable size would be of significant benefit to the tomato industry. Further, such tomato lines may also enable machine harvesting, reducing the dependence on farm labor.

Introduction of the brachytic trait into normal phenotype tomatoes resulted in tomatoes with shortened internodes. Since the introduction of brachytic (br) into fresh-market tomato breeding programs in 1980s, the locus has been shown to be the primary source of the shortened internode phenotype. It is notable that no evidence for a significant negative correlation observed between marketable fruit harvests and the br has been reported in a peer-reviewed forum. Identification of genes or mutations that results in plants with shortened stem length

A reduced plant height driven by shortened stems is beneficial for improving crop yield potential. The presence of br is an important consideration in developing tomatoes intended for mechanical harvest. There is a need to breed new genes that optimize phenotypes for such mechanization into fresh-market adapted tomato cultivars.

### SUMMARY

Regulation of stem length is an important target trait in plant breeding and genetics. Described are tomato brachytic loci that control stem length. Disruption of these brachytic loci result in plants having shortened internode length. Described are compositions and methods for generating plants having shortened internode length.

Described are loci responsible for the brachytic phenotype in plants of the family Solanaceae (brachytic locus). The loci are open reading frames located at Solyc01g066950, Solyc01g066970, Solyc06g005530, and Solyc12g099610 of S. lycopersicum. Solanaceae plants homozygous for loss of function alleles at one or more of these loci have shortened internode length. In some embodiments, Solanaceae plants heterozygous for loss of function alleles at one or more of these loci may have shortened internode length.

Described are CRISPR constructs and systems that can be used to generate brachytic Solanaceae plants rapidly and efficiently. A brachytic phenotype can be introduced into a Solanaceae plant having one or more other desired traits by using the described CRISPR constructs and systems to generate loss of function mutations in one or more brachytic loci in the desired plant. The described CRISPR constructs and systems can be used to introduce a loss of function mutation at one or more of the open reading frames located at Solyc01g066950, Solyc01g066970, Solyc06g005530, and Solyc12g099610. The described CRISPR constructs can be further combined with a CRISPR construct or system for introducing a loos of function mutation in an open reading frame located at Solyc01g066980.

In some embodiments, the CRISPR constructs are used to introduce a mutant brachytic allele into a Solanaceae plant. The modified plants is then used to introgress the brachytic allele into other genetic backgrounds. The resultant plants have shortened internodes. The shortened internodes lead to shorter plants that do not require staking.

The methods can be used to introduce a brachytic phenotype into a Solanaceae plant having a desired characteristic, such as fruit size, fruit number and/or fruit quality. In some embodiments, the brachytic plants do not require staking. In some embodiments, the brachytic plants provide a suitable plant habit for machine harvest. Normal tomato plants may require tying 3-4 times per season. Having shorter tomato plants reduces tying cost (materials & labor costs) under current horticultural practices/cultivation systems. In some embodiments, the described brachytic plants are tied, 0, 1, or 2 times per year. In some embodiments, the described brachytic plants require fewer tyings than normal plants. In some embodiments, the number of tyings of the described brachytic plants during the season is reduced by 1, 2, 3, or 4 times compared to normal plants without the brachytic mutations/disruptions.

CRISPR constructs and systems for directed modification (disruption) of one or more brachytic loci in Solanaceae are described. The modification can be a deletion, a missense mutation, a nonsense mutation, an insertion mutation of a combination of these.

In some embodiments the CRISPR constructs and systems are used to generate genetically modified Solanaceae plants carrying a one or more loss of functions brachytic loci alleles and having a brachytic phenotype. The transgenic plants can then be used to produce progeny brachytic plants. Any of the described CRISPR constructs and systems can be used to generate a transgenic Solanaceae plant carrying a loss of function brachytic locus allele. The described CRISPR constructs and systems can be used to introduce loss of function mutations in one or more of the reading frames located at Solyc01g066950, Solyc01g066970, Solyc06g005530, and Solyc12g099610. The described CRISPR constructs can be further combined with a CRISPR construct or system for introducing a loss of function mutation into an open reading frame located at Solyc01g066980. The CRISPR constructs and systems can be used to introduce loss of function mutations into two or more reading frames simultaneously, sequentially, or a combination thereof

A Solanaceae plant can be a S. Solanum or a Capsicum plant. A Solanum plant can be a S. melongena (eggplant) plant, a S. tuberosum (potato) plant, or a tomato plant. A Capsicum plant can be a C. annuum (pepper) plant or a C. frutescens (tabasco pepper) plant. The term tomato includes but is not limited to any species of tomato. In some embodiments, tomato plant can be a Solanum lycopersicum plant, a S. pimpinellifolium plant, or a S. pennellii plant. In some embodiments, the tomato plant is a Solanum lycopersicum plant.

In some embodiments, methods of producing brachytic plants and methods of genetically modifying a plant to produce a brachytic plant using a Clustered Regularly Interspaced Short Palindromic Repeats (CRISPR)/CRISPR-associated (Cas) system are described. In some embodiments, brachytic plants created using a CRISPR system are described. In some embodiments, nucleic acids for producing a brachytic plant using a CRISPR system are described.

### BRIEF DESCRIPTION OF THE DRAWINGS

FIG. 1. Illustration showing crRNA guide sequences for modification of the Solyc01g066970 and Solyc01g066950 loci. Mutations in the Solyc01g066970 and Solyc01g066950 loci generated using CRISPR systems with gRNAs having the indicated guide sequences are also shown.

FIG. 2. CRISPR!Cas9-driven single mutant (brachytic) plant (left), which shows a shortened internode length compared to its background Fla 8059 (right). Scale bar=10 cm.

FIG. 3. Graph illustrating reduced stem length in double-mutant plants. White bar =wild type plants. Dark bar=br0.5CRbr.7.2CR (M1) plants. Statistically significant ***P<0.001 based on a two-tailed t-test.

FIG. 4. Network analysis of gene expression patterns across tissues, genotypes, and gibberellic acid (GA) treatments. (A) Diagram illustrating phylogenetic tree of Solanaceae flowering promoting factor 1 (FPF1) families. Dots represent five modern tomato (Solanum lycopersicum) FPF1s identified by sequence similarity to the families in Solanaceae species. Wild tomatoes (S. pimpinellifolium and S. pennellii) are indicated by asterisks. Scale bar represents 1.0 substitutions per site. (B) Graph illustrating expression of tomato FPF1s in different tissues. WT=wild-type plant, M=br plant (Solyc01g066980). For each expression levels are indicated, in order, for Solyc01g066950, Solyc01g066970, Solyc01g066990, Solyc06g005530, and Solyc12g099610.

FIG. 5. Diagram illustrating two flowering promoting factor 1 (FPF1) genes (Solyc01g066950 and Solyc01g066970), the centromere-proximal homologs of brachytic. A CRISPR-Cas9 system utilizing a single-guide RNA that targeted a sequence region with only a single nucleotide difference (boxed) between the two homologous FPF1s (i.e., “A” at 68,005,223 bp on Solyc01g066950 and “G” at 68,057,560 bp on Solyc01g066970) as used to generate loss of function mutations. The first nucleotide position of the each start codon is given. Sequences of three different mutants (br.7^{CR}, br.57.1^{CR}, br.57.2^{CR}) are shown. Deletions and insertions are indicated by blue dashes and underlines, respectively. The sequence gap length between two genes is shown in parentheses. WT=wild-type.

| | | | SEQ ID |
|---|---|---|---|
| Plant | Allele | Sequence | NO: |
| WT | Solyc01g066950 | CCGTCGCACCGTG | 107 |
| | | AAAGTCACCGAGG | |
| | Solyc01g066970 | CCGTCGCACCGTG | 108 |
| | | GAAGTCACCGGGG | |
| br.7^{CR} | Solyc01g066950 | CCGTCGCACCGTG | 109 |
| | | AAAGTCACCGAGG | |
| | Solyc01g066970 | CCGTCGCAACCGT | 110 |
| | | GGAAGTCACCGGG | |
| | | G | |
| br.57.1^{CR} | Solyc01g066950 | CCGTCGCACCGTG | 111 |
| | | AACCGAGG | |
| | Solyc01g066970 | CCGTCGCACCGTG | 112 |
| | | GACCGGGG | |
| br.57.2^{CR} | Solyc01g066950 | CCGTCGCACCGTG | 113 |
| | | AAAGTCAACCGAG | |
| | | G | |
| | Solyc01g066970 | CCGTCGCACCGTG | 114 |
| | | GACCGGGG | |

FIG. 6. Graph illustrating reduced plant height in plants harboring mutated brachytic homologs at Solyc01g066950 and Solyc01g066970. Stem lengths of 6-week-old plants are shown. Mutants are transgene-free, homozygous M2 generation. The n value represents the total number of plants for each genotype evaluated. **p<0.01 based on one-way ANOVA in conjunction with a two-tailed Tukey's HSD multiple comparison test. Error bars indicate 95% confidence intervals.

### DETAILED DESCRIPTION

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition, 2006). As appropriate, procedures involving the use of commercially available kits and reagents are generally carried out in accordance with manufacturer defined protocols and/or parameters unless otherwise noted.

The use of “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” indicates within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

The term “nucleic acid” refers to deoxyribonucleotides or ribonucleotides and polymers thereof (“polynucleotides”) in either single- or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection.

The term “plant” includes whole plants, plant organs (e.g., leaves, stems, flowers, roots, reproductive organs, embryos and parts thereof, etc.), seedlings, seeds and plant cells and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous.

“Early flowering” refers to increasing the ability of the plant to exhibit early flowering as compared to a matching control plant (e.g., a similar plant not having the brachytic phenotype). In some embodiments, early flowering indicates a shorter time period between germination to the time in which the first flower opens. In some embodiments, increasing early flowering of a population of plants increases the number or percentage of plants having an early flowering. In some embodiments, early flowering enables the plant to produce more flowers, fruits, pods and seeds without changing plant maturity period. Early flowering can also lead to increased yield by providing a longer grain filling or fruit maturation period.

The term “locus” refers to a position on the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

“Introgression” or “introgressing” of a brachytic locus means introduction of a brachytic locus from a donor plant comprising the brachytic locus into a recipient plant by standard breeding techniques, wherein selection can be done phenotypically by means of observation of the internodal length or plant height, or selection can be done with the use of brachytic markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. Selection is started in the F1 or any further generation from a cross between the recipient plant and the donor plant, suitably by using markers as identified herein. The skilled person is however familiar with creating and using new molecular markers that can identify or are linked to the brachytic locus.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs typically retain the same function in the course of evolution. “Paralogous” genes include genes related by duplication within a genome. Paralogs can evolve new functions in the course of evolution.

Compositions or methods “comprising” or “including” one or more recited elements may include other elements not specifically recited. For example, a composition that “comprises” or “includes” a marker may contain the marker alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”). The term “or” refers to any one member of a particular list and also includes any combination of members of that list.

The singular forms of the articles “a,” “an,” and “the” include plural references unless the context clearly dictates otherwise. For example, the term “a marker” or “at least one marker” can include a plurality of markers, including mixtures thereof.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “regenerant” is a plant produced from a plant tissue cell, such as a genetically modified plant tissue cell.

### II. Overview

Described are compositions, including CRISPR constructs, for modifying one or more brachytic loci in a plant and methods of using the compositions for producing plants having a brachytic phenotype (i.e., brachytic plants). In some embodiments, the plant is a Solanaceae plant A Solanaceae plant can be, but is not limited to, a Solanum or a Capsicum plant. A Solanum plant can be, but is not limited to, a S. melongena (eggplant) plant, S. tuberosum (potato) plant, or a tomato plant. A Capsicum plant can be, but is not limited to, a C. annuum (pepper) plant or a C. frutescens (tabasco pepper) plant. In some embodiments, the Solanaceae plant is a tomato plant. The term tomato is not limited to any species or variety of tomato. In some embodiments, tomato plant can be a Solanum lycopersicum plant, a S. pimpinellifolium plant, or a S. pennellii plant. In some embodiments, the tomato plant is a Solanum lycopersicum plant.

In some embodiments, the brachytic loci are homologs of the Br gene located at Solyc01g066980 (also termed flowering promoting factor 1 or FPF1).

In some embodiments, nucleic acids for producing brachytic plants using CRISPR systems are described. The CRISPR systems can target one or more of the brachytic loci. The nucleic acids include, but are not limited to, nucleic acids comprising crRNAs or gRNAs and nucleic acids encoding crRNAs or gRNAs.

In some embodiments, methods of producing brachytic Solanaceae plants and methods of genetically modifying a Solanaceae plant to produce a brachytic plant using a CRISPR system are described.

In some embodiments, Solanaceae plants having a brachytic phenotype produced using any one or more of the described CRISPR constructs are described.

A “brachytic plant” is characterized by having shortened internodes without a substantial corresponding reduction in the number of size of other plant parts (brachytic phenotype). Shortened internodes drive shortened stem length/plant height compared to normal plants. Brachytic (shortened) internodes are distinguishable from a dwarf-mediated phenotype in which all parts are shortened. In some embodiments, the brachytic plants also have accelerated or early flowering.

A “brachytic locus” comprises a locus that corresponds to the brachytic measurable trait (phenotype). Plants homozygous for a loss of function mutation at a brachytic locus exhibit the brachytic phenotype, i.e., the plants have a shorter internode length compared to otherwise genetically similar plants that are not homozygous for the loss of function mutation at the brachytic locus. Plants homozygous for a wild-type gene at a brachytic locus exhibit normal growth with respect to the brachytic phenotype. Plants heterozygous at the brachytic locus, carrying one wild-type brachytic allele and one loss of function brachytic allele, may exhibit intermediate growth characteristics with respect to the brachytic phenotype. Brachytic loci include homologs and paralogs of SEQ ID NO: 21 or 22 (Solyc01g066980 locus) in tomato plants and orthologs thereof in other Solanaceae plants. In some embodiments, a brachytic locus is selected from the group consisting of: a Solyc01g066950 locus, a Solyc01g066970 locus, a Solyc06g005530 locus, and a Solyc12g099610 locus, and orthologs thereof.

A “Solyc01g066950 locus” comprises Solyc01g066950.1.1: SEQ ID NO: 2 (DNA).

A “Solyc01g066970 locus” comprises Solyc01g066970.2.1: SEQ ID NO: 7 (DNA).

A “Solyc06g005530 locus” comprises Solyc06g005530.2.1: SEQ ID NO: 12 (DNA).

A “Solyc12g099610 locus” comprises Solyc12g099610.1.1: SEQ ID NO: 17 (DNA).

A “Solyc01g066980 locus” comprises Solyc01g066980.2.1: SEQ ID NO: 102 (DNA).

In some embodiments, the brachytic locus includes sequence 5′ and/or 3′ of the coding sequence. In some embodiments, a “Solyc01g066950 locus” comprises Solyc01g066950.1.1: SEQ ID NO: 1 (DNA). In some embodiments, a “Solyc01g066970 locus” comprises Solyc01g066970.2.1: SEQ ID NO: 6 (DNA). In some embodiments, a “Solyc06g005530 locus” comprises Solyc06g005530.2.1: SEQ ID NO: 11 (DNA). In some embodiments, a “Solyc12g099610 locus” comprises Solyc12g099610.1.1: SEQ ID NO: 16 (DNA). In some embodiments, a “Solyc01g066980 locus” comprises Solyc01g066980.2.1: SEQ ID NO: 102 (DNA; US202010045901).

The described brachytic loci can be targeted to genetically modify Solanaceae plants to yield a brachytic phenotype. Solanaceae plants having a loss of function mutation in both alleles (homozygous plants) of one or more of the brachytic loci have shortened internodes compared to the otherwise genetically identical plants homozygous for wild-type alleles and the brachytic loci. Solanaceae plants having a loss of function mutation in one alleles (heterozygous plants) of one or more of the brachytic loci may have shortened internodes compared to the otherwise genetically identical plants homozygous for wild-type alleles and the brachytic loci.

### III. CRISPR Systems

Described are nucleic acids for producing brachytic plants using a CRISPR (e.g., CRISPR/Cas) system are described. The described nucleic acids can be used to target modification/mutation of one or more brachytic loci in a plant.

A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system can be delivered to a plant or plant cell via a bacterium. The bacterium can be, but is not limited to, Agrobacterium tumefaciens.

The CRISPR system is designed to target one or more of the described brachytic loci. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

Guide sequences suitable for forming gRNAs or crRNAs for CRISPR system mediated genetic modification of a brachytic locus are described. Suitable guide sequences include 17-20 nucleotide sequences in any of SEQ ID NOs: 1, 2, 6, 7, 11, 12, 16, 17, 21, and 102 or a complement thereof that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in SEQ ID NO: 1, 2, 6, 7, 11, 12, 16, 17, 21, and 102 or a complement thereof can be used in forming a gRNA. zCas9 PAM sites in SEQ ID NOs: 1, 2, 6, 7, 11, 12, 16, 17, 21, and 102, GG and CC, are shown in bold capital letters (Table 1). CC sequences in the listed strand correspond to GG sequences in the complementary strand. Deletions or insertions in the flanking regions may alter expression of the gene leading to plants displaying a brachytic phenotype. In some embodiments, the guide sequence is 100% complementary to the target sequence. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence.

CRISPR modification of a brachytic locus is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophiles), and NAAAAC (Treponema denticola). Guide sequences for CRISPR systems having nucleases with different PAM sequence requirements are identified as described above for zCas9, substituting the different PAM sequences.

In some embodiments, the CRISPR system comprises one or more RNA-guided DNA endonucleases or one or more nucleic acids encoding the one or more RNA-guided DNA endonuclease, and one or more of:
(a) a guide RNA comprising a 17-20 nucleotide guide sequence comprising 17-20 contiguous nucleotides from SEQ ID NO: 1 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(b) a guide RNA comprising a 17-20 nucleotide guide sequence comprising 17-20 contiguous nucleotides from SEQ ID NO: 6 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(c) a guide RNA comprising a 17-20 nucleotide guide sequence comprising 17-20 contiguous nucleotides from SEQ ID NO: 11 differing by no more than 1 or 2 nucleotides, or a complement thereof, wherein the 17-20 nucleotide sequence is unique compared to the rest of the genome and immediately adjacent (5′) to a protospacer-adjacent motif (PAM) site; and
(d) a guide RNA comprising a 17-20 nucleotide guide sequence comprising 17-20 contiguous nucleotides from SEQ ID NO: 16 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system further comprises a guide RNA comprising a 17-20 nucleotide guide sequence comprising 17-20 contiguous nucleotides from SEQ ID NO: 21 or 102 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system comprises one or more RNA-guided DNA endonucleases or one or more nucleic acids encoding the one or more RNA-guided DNA endonuclease, and one or more of:
(a) a guide RNA comprising a 17-20 nucleotide guide sequence comprising 17-20 contiguous nucleotides from SEQ ID NO: 2 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(b) a guide RNA comprising a 17-20 nucleotide guide sequence comprising 17-20 contiguous nucleotides from SEQ ID NO: 7 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(c) a guide RNA comprising a 17-20 nucleotide guide sequence comprising 17-20 contiguous nucleotides from SEQ ID NO: 12 differing by no more than 1 or 2 nucleotides, or a complement thereof, wherein the 17-20 nucleotide sequence is unique compared to the rest of the genome and immediately adjacent (5′) to a protospacer-adjacent motif (PAM) site; and
(d) a guide RNA comprising a 17-20 nucleotide guide sequence comprising 17-20 contiguous nucleotides from SEQ ID NO: 17 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system further comprises a guide RNA comprising a 17-20 nucleotide guide sequence comprising 17-20 contiguous nucleotides from SEQ ID NO: 21 or 102 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system comprises one or more RNA-guided DNA endonucleases or one or more nucleic acids encoding the one or more RNA-guided DNA endonuclease, and one or more of:
(a) one or more guide RNAs each comprising a 17-20 nucleotide guide sequence comprising 17-20 contiguous nucleotides from SEQ ID NO: 1 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(b) one or more guide RNAs each comprising a 17-20 nucleotide guide sequence comprising 17-20 contiguous nucleotides from SEQ ID NO: 6 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(c) one or more guide RNAs each comprising a 17-20 nucleotide guide sequence comprising 17-20 contiguous nucleotides from SEQ ID NO: 11 differing by no more than 1 or 2 nucleotides, or a complement thereof, wherein the 17-20 nucleotide sequence is unique compared to the rest of the genome and immediately adjacent (5′) to a protospacer-adjacent motif (PAM) site; and
(d) one or more guide RNAs each comprising a 17-20 nucleotide guide sequence comprising 17-20 contiguous nucleotides from SEQ ID NO: 16 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system further comprises a guide RNA comprising a 17-20 nucleotide guide sequence comprising 17-20 contiguous nucleotides from SEQ ID NO: 21 or 102 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system comprises one or more RNA-guided DNA endonucleases or one or more nucleic acids encoding the one or more RNA-guided DNA endonuclease, and one or more of:
(a) one or more guide RNAs each comprising a 17-20 nucleotide guide sequence comprising 17-20 contiguous nucleotides from SEQ ID NO: 2 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(b) one or more guide RNAs each comprising a 17-20 nucleotide guide sequence comprising 17-20 contiguous nucleotides from SEQ ID NO: 7 differing by no more than 1 or 2 nucleotides, or a complement thereof, wherein the 17-20 nucleotide sequence is unique compared to the rest of the genome and immediately adjacent (5′) to a protospacer-adjacent motif (PAM) site;
(c) one or more guide RNAs each comprising a 17-20 nucleotide guide sequence comprising 17-20 contiguous nucleotides from SEQ ID NO: 12 differing by no more than 1 or 2 nucleotides, or a complement thereof, wherein the 17-20 nucleotide sequence is unique compared to the rest of the genome and immediately adjacent (5′) to a protospacer-adjacent motif (PAM) site; and
(d) one or more guide RNAs each comprising a 17-20 nucleotide guide sequence comprising 17-20 contiguous nucleotides from SEQ ID NO: 17 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system further comprises a guide RNA comprising a 17-20 nucleotide guide sequence comprising 17-20 contiguous nucleotides from SEQ ID NO: 21 or 102 differing by no more than 1 or 2 nucleotides, or a complement thereof, wherein the 17-20 nucleotide sequence is unique compared to the rest of the genome and immediately adjacent (5′) to a protospacer-adjacent motif (PAM) site.

In some embodiments, the CRISPR system comprises one or more guide RNAs selected from the group consisting of: a guide RNA comprising SEQ ID NO: 5, a guide RNA comprising SEQ ID NO: 9, a guide RNA comprising SEQ ID NO: 10, a guide RNA comprising SEQ ID NO: 14, a guide RNA comprising SEQ ID NO: 15, a guide RNA comprising any one of SEQ ID NO: 76-92, a guide RNA comprising SEQ ID NO: 19, a guide RNA comprising SEQ ID NO: 20, and a guide RNA comprising any one of SEQ ID NO: 92-101. The sequences in Table 1 are listed as DNA sequences. It is understood that RNA equivalents of the listed DNA sequences, substituting uracils (U) for thymines (T), may be used. An “RNA equivalent” is an RNA molecule having essentially the same complementary base pair hybridization properties as the listed DNA sequence.

In some embodiments, the CRISPR system further comprises a guide RNA comprising TCTAGTGGAGAACTCCGAT (SEQ ID NO: 103; wherein T's can be U's), a guide RNA comprising AAAAGTTCTTGTACATCTTC (SEQ ID NO: 104; wherein T′s can be U′s), or a guide RNA comprising SEQ ID NO: 103 and a guide RNA comprising SEQ ID NO: 104.

In some embodiments, the CRISPR system comprises one or more guide sequences selected from the group consisting of: a guide RNA comprising SEQ ID NO: 5, a guide RNA comprising SEQ ID NO: 9, a guide RNA comprising SEQ ID NO: 10, a guide RNA comprising SEQ ID NO: 14, a guide RNA comprising SEQ ID NO: 15, a guide RNA comprising any one of SEQ ID NO: 76-92, a guide RNA comprising SEQ ID NO: 19, a guide RNA comprising SEQ ID NO: 20, and a guide RNA comprising any one of SEQ ID NO: 92-101. It is understood that RNA equivalents of the listed DNA sequences, substituting uracils (U) for thymines (T), may be used. An “RNA equivalent” is an RNA molecule having essentially the same complementary base pair hybridization properties as the listed DNA sequence.

In some embodiments, the CRISPR system further comprises a guide RNA comprising a 17-20 nucleotide guide sequence comprising 17-20 contiguous nucleotides from SEQ ID NO: 21 or 102 differing by no more than 1 or 2 nucleotides, or a complement thereof, wherein the 17-20 nucleotide guide sequence is unique compared to the rest of the genome and immediately adjacent (5′) to a protospacer-adjacent motif (PAM) site.

Two or more guide RNAs can used with the same RNA-guided DNA endonuclease (e.g., Cas nuclease) or different RNA-guided DNA endonucleases.

In some embodiments, two or more gRNAs targeting two or more different brachytic loci are used. The two or more gRNAs can be used with the same RNA-guided DNA endonuclease or different RNA-guided DNA endonucleases.

In some embodiments, three or more gRNAs targeting three or more different brachytic loci are used. The three or more gRNAs can used with the same RNA-guided DNA endonuclease or different RNA-guided DNA endonucleases.

In some embodiments, four or more gRNAs targeting four or more different brachytic loci are used. The four or more gRNAs can used with the same RNA-guided DNA endonuclease or different RNA-guided DNA endonucleases.

In some embodiments, five or more gRNAs targeting five or more different brachytic loci are used. The five or more gRNAs can used with the same RNA-guided DNA endonuclease or different RNA-guided DNA endonucleases.

In some embodiments, two or more gRNAs targeting a single brachytic locus can be used. The two or more gRNAs can used with the same RNA-guided DNA endonuclease (Cas nuclease) or different RNA-guided DNA endonucleases.

It is noted that, for RNA sequences, T′s of SEQ ID NO: 1, 2, 6, 7, 11, 12, 16, 17, 21, and 102 can be U's. In some embodiments, the PAM site is 5′-NGG-3′.

Guide RNAs for modification of brachytic loci in other Solanaceae plants are generated in a similar manner by identifying the corresponding ortholog sequences of the Solyc01g066950 locus, the Solyc01g066970 locus, the Solyc06g005530 locus, and/or the Solyc12g099610 locus in the other Solanaceae plants and selecting target sequences as described above. Exemplary orthologs of brachytic loci as shown in Tables 2A-F.

Any of the above described guide RNAs can be provided as an RNA or a DNA encoding the RNA.

In some embodiments, a CRISPR system comprises one or more guide RNAs and a nucleic acid encoding an RNA-guided DNA endonuclease.

In some embodiments, a CRISPR system comprises one or more guide RNAs and a one or more nucleic acids encoding two or more different RNA-guided DNA endonucleases.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease.

### IV. CRISPR-Modified Plants

Methods of producing brachytic plants and methods of genetically modifying a plant to produce a brachytic plant using a CRISPR system are described.

Described are methods of generating genetically modified brachytic plants comprising introducing into a plant, a plant tissue, or a plant cell, one or more of the described CRISPR systems. In some embodiments, genetically modified brachytic plants created using a CRISPR system are described. In some embodiments, the CRISPR system is a CRISPR/Cas system.

In some embodiments, methods are described for producing a brachytic tomato plant, the methods comprising the steps of: a) introducing into the plant one or more of the described CRISPR systems. In some embodiments, at least two CRISPR guide RNA's are used.

Nucleic acids may be introduced into a plant cell or cells using a number of methods known in the art, including but not limited to electroporation, DNA bombardment or biolistic approaches, microinjection, via the use of various DNA-based vectors such as Agrobacterium tumefaciens and Agrobacterium rhizogenes vectors, and CRISPR or CRISPR/Cas9. Once a plant cell has been successfully transformed, it may be cultivated to regenerate a transgenic plant (regenerant).

Various methods for introducing the transgene expression vector constructs of the invention into a plant or plant cell are well known to those skilled in the art, and any method capable of transforming the target plant or plant cell may be utilized.

In some embodiments, Agrobacterium tumefaciens is used to deliver CRISP system nucleic acids to a plant. Agrobacterium-mediated transformation of a large number of plants are extensively described in the literature (see, for example, Agrobacterium Protocols, Wan, ed., Humana Press, 2^{nd} edition, 2006). Various methods for introducing DNA into Agrobacteria are known, including electroporation, freeze/thaw methods, and triparental mating. In some embodiments, a pMON316-based vector is used in the leaf disc transformation system of Horsch et al. Other commonly used transformation methods include, but are not limited to, microprojectile bombardment, biolistic transformation, and protoplast transformation of naked DNA by calcium, polyethylene glycol (PEG) or electroporation (Paszkowski et al., 1984, EMBO J. 3: 2727-2722; Potrykus et al., 1985, Mol. Gen. Genet. 199: 169-177; Fromm et al., 1985, Proc. Nat. Acad. Sci. USA 82: 5824-5828; Shimamoto et al., 1989, Nature, 338: 274-276.

T₀ transgenic plants may be used to generate subsequent generations (e.g., T₁, T₂, etc.) by selfing of primary or secondary transformants, or by sexual crossing of primary or secondary transformants with other plants (transformed or untransformed).

The described CRISPR systems can be used to genetic modify one or more brachytic loci in a plant. The plant can be a plant having a trait of interest. Delivery of the CRISPR system leads to small nucleotide insertions or deletions in or near the target sequence, resulting in disruption of the targeted brachytic locus. Introducing a brachytic phenotype into a plant having a desired trait may result in a cost savings for plant developers, because such methods eliminate traditional plant breeding. A disruption is a modification, such as a deletion, a missense mutation, a nonsense mutation, an insertion mutation of a combination of these, that results in a loss of function of the locus or protein encoded by the locus or reduced expression of the locus or protein encoded by the locus. In some embodiments, the disruption comprises a deletion. In some embodiments, the deletion comprises a 1-10 nucleotide or base pair deletion. In some embodiments, the deletion comprises a 1-5 nucleotide or base pair deletion. In some embodiments, the deletion comprises a 1, 2, 3, 4, 5, 6, 7, 8, 9, or 10 nucleotide or base pair deletion.

In some embodiments, the described CRISPR systems can be used to genetic modify 1, 2, 3, 4, or 5 brachytic loci in a plant.

In some embodiments, the described CRISPR constructs may be used to introduce one or more determinants of brachytic into a Solanaceae plant by genetic transformation.

In some embodiments, the CRISPR system is modify one or more brachytic loci into a transgenic tomato line. The transgenic tomato line can contain one or more genes for herbicide tolerance, increased yield, insect control, fungal disease resistance, virus resistance, bacterial disease resistance, germination and/or seedling growth control, enhanced animal and/or human nutrition, improved processing traits, or improved flavor, among others.

Plants produced using the described CRISPR systems (having loss of function mutations in one or more brachytic homolog loci) have a brachytic phenotype. The brachytic plants can produce similar sizes and quantities of fruit to an otherwise genetically similar plants lacking the loss of function mutations in the one or more brachytic homolog loci. In some embodiments, the brachytic plants produce fruits at a yield of greater than 50%, greater than 60%, greater than 70%, greater than 80%, or greater than 90% of the yield of an otherwise genetically similar plant lacking the loss of function mutation in one or more brachytic loci when grown under the same conditions. In some embodiments, the brachytic plants produce fruits having an average size that is greater than 50%, greater than 60%, greater than 70%, greater than 80%, or greater than 90% of the average size of fruits produced by an otherwise genetically similar plant lacking the loss of function mutation in one or more brachytic loci when grown under the same conditions. In some embodiments, the brachytic plants produce fruits having an average weight that is greater than 50%, greater than 60%, greater than 70%, greater than 80%, or greater than 90% of the average weight of fruits produced by an otherwise genetically similar plant lacking the loss of function mutation in one or more brachytic loci when grown under the same conditions. In some embodiments, the brachytic plants produce greater than 50%, greater than 60%, greater than 70%, greater than 80%, or greater than 90% of the number of medium size or larger fruits per plant compared to the number of medium size or larger fruits per plant produced by an otherwise genetically similar plant lacking the loss of function mutation in one or more brachytic loci when grown under the same conditions. In some embodiments, the brachytic plants produce greater than 50%, greater than 60%, greater than 70%, greater than 80%, or greater than 90% of the number of large or extra large size fruits per plant compared to the number of large or extra large size fruits per plant produced by an otherwise genetically similar plant lacking the loss of function mutation in one or more brachytic loci when grown under the same conditions.

### Tomato Fruit Size

| | Diameter in inches | Weight in ounces |
|---|---|---|
| Size | (mm) | (grams) |
| Small | $2 \frac{1}{8} - 2 \frac{9}{32}$ (53.98-57.94) | <3 oz (<85) |
| Medium | $2 \frac{1}{4} - 2 \frac{17}{32}$ (57.15-64.29) | 3-6 oz (85-170) |
| Large | $2 \frac{1}{2} - 2 \frac{25}{32}$ (63.5-70.64) | >6 to 10 oz (>170-283) |
| Extra Large | $\underline{>} 2 \frac{3}{4}$ (69.85) | >10 oz (>283) |

### V. Sequences

The nucleotide and amino acid sequences listed in the accompanying sequence listing are shown using standard letter abbreviations for nucleotide bases, and single-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

### VI. Detection of a Modified Gene

Modification of a brachytic locus using any of the described CRISPR constructs can be detected or confirmed by any means known in the art for detecting genetic modifications.

In some embodiments, a modification can be detected in genomic DNA sample. Genomic DNA samples include, but are not limited to, genomic DNA isolated directly from a plant, cloned genomic DNA, or amplified genomic DNA.

Genetic analysis methods include, but are not limited to, polymerase chain reaction (PCR)-based detection methods (for example, TaqMan assays), microarray methods, mass spectrometry-based methods and/or nucleic acid sequencing methods, including whole genome sequencing. In some embodiments, the detection of genetic modification in a sample of DNA, RNA, or cDNA may be facilitated through the use of nucleic acid amplification methods. Such methods specifically increase the concentration of polynucleotides that span a target site, or include that site and sequences located either distal or proximal to it. Such amplified molecules can be readily detected by gel electrophoresis, fluorescence detection methods, or other means.

In some embodiments, a brachytic locus genetic modification is detected by hybridization to allele-specific oligonucleotide (ASO) probes. ASO probes are disclosed in U.S. Pat. Nos. 5,468,613 and 5,217,863. 5,468,613. Single or multiple nucleotide variations in nucleic acid sequence can be detected in nucleic acids by a process in which the sequence containing the nucleotide variation is amplified, spotted on a membrane and treated with a labeled allele-specific oligonucleotide probe.

In some embodiments, a brachytic locus genetic modification is detected by probe ligation methods. Probe ligation methods disclosed in U.S. Pat. No. 5,800,944 where sequence of interest is amplified and hybridized to probes followed by ligation to detect a labeled part of the probe.

In some embodiments, microarrays can be used for detection of brachytic locus genetic modification. For microarray detection, oligonucleotide probe sets are assembled in an overlapping fashion to represent a single sequence such that a difference in the target sequence at one point would result in partial probe hybridization (Borevitz et al., Genome Res. 13:513-523, 2003; Cui et al., Bioinformatics 21:3852-3858, 2005). Typing of target sequences by microarray-based methods is disclosed in U.S. Pat. Nos. 6,799,122; 6,913,879; and 6,996,476.

In some embodiments, a brachytic locus genetic modification can be directly identified or sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

In some embodiments, the presence of a brachytic marker in a plant may be detected through the use of a nucleotide probe. A probe may be, but is not limited to, nucleotide molecule, polynucleotide, oligonucleotide, DNA molecule, RNA molecule, PNA, UNA, locked nucleotide, or modified polynucleotide. Polynucleotides can be synthesized by any means known in the art. A probe may contain all or a portion of the nucleotide sequence of the genetic marker and optionally, one or more additional sequences. The one or more additional sequences can be contiguous nucleotide sequence from the plant genome, non-contiguous nucleotide sequence from the plant genome, or sequence that is not from the plant genome. Additional, contiguous nucleotide sequence can be “upstream” or “downstream” of the original marker, depending on whether the contiguous nucleotide sequence from the plant chromosome is on the 5′ or the 3′ side of the original marker, as conventionally understood. As is recognized by those of ordinary skill in the art, the process of obtaining additional, contiguous nucleotide sequence for inclusion in a marker may be repeated nearly indefinitely (limited only by the length of the chromosome), thereby identifying additional markers along the chromosome.

A polynucleotide probe may be labeled or unlabeled. A wide variety of techniques are readily available in the art for labeling a nucleotide probe. Nucleotide labels include, but are not limited to, radiolabeling, fluorophores, haptens, antibodies, antigens, enzymes, enzyme substrates, enzyme cofactors, and enzyme inhibitors. A label may provide a detectable signal by itself (e.g., a radiolabel or fluorophore) or in conjunction with other agents.

A probe may be an exact copy of a marker to be detected. A probe may also be a nucleic acid molecule comprising, or consisting of, a nucleotide sequence which is substantially identical to a cloned segment of the Solanaceae chromosomal DNA. The term “substantially identical” may refer to nucleotide sequences that are more than 85% identical. For example, a substantially identical nucleotide sequence may be 86%, 87%, 88%, 89%, 90%, 91%, 92%, 93%, 94%, 95%, 96%, 97%, 98%, or 99% identical to the reference sequence.

A probe may also be a nucleic acid molecule that is “specifically hybridizable” or “specifically complementary” to an exact copy of the marker to be detected (“DNA target”). “Specifically hybridizable” and “specifically complementary” are terms that indicate a sufficient degree of complementarity such that stable and specific binding occurs between the nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired. Thus, an oligonucleotide probe is “specifically hybridizable” to a maker allele if stable and specific binding occurs between the oligonucleotide probe and the marker allele (e.g., a SNP marker) under stringent hybridization conditions, but stable and specific binding does not occur between the oligonucleotide probe and the wild-type allele at the marker position.

In some embodiments, a probe comprises a pair primers designed to produce an amplification product, wherein the amplification product is directly or indirectly determinative for the presence or absence of a brachytic marker

TABLE 1
CRISPR modification of tomato plants - sequences (underlined sequence = open

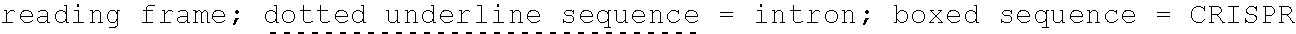

target sequence; bold capital letters = zCas9 PAM sites). It is
understood that RNA equivalents of the listed DNA sequences,
substituting uracils (U) for thymines (T), may be used.

Solyc01g066950 locus SEQ ID NO: 1 (5′→3′)
aatatactcaatctaatgaaCCtaattCCcaaatgagtatGGtattgaGGcttgagtCCtcatgtgtgaacttGGcG
Gtacttattaacgatcatagtacttgttgttgctacatgttgagtaatgtagttgatttcatattattacttgatat
atattgctttctattttgagttGGCCgatgatcgtgttttgtactgaCCCCtacttgtatgtttctttCCttgttat
ttgtGGagtgcagcaaacgtgCCgtcgtctttaactcaaCCgcaactctagCCgatcttcattacaCCGGatttcaG
GGtgagctaacgcttctagcttGGactGGatcttcttcttcatgtctcgatgCCttgaagttCCGGcatgaactagc
ttttatttattctagctttctagatactcttagctttagtaatttgaGGatagatgttcttatgatgatgacttCCa
gattttGGGGataataatagttgttgagtttttagaagttatttaattgattttcattaatgaGGttaagtcttCCg
cattatattCCgtcattatattgaaatgttGGGtttagattGGttGGttcgctcacataGGaagataaatgtGGGtg
CCactcgcGGtCCgttttGGGtcgtgacaGGtaaattaGGGtatcttgtGGCCatataaatattctCCCtttctttt
tctttaatcttatgagcgtacgataagttagtataattctaaatCCtaCCtattaatcatcatcaattttattaaat
aagaaagaaaatactttttgCCaCCtaatgtattttttattacatagaaaCCCgtataaaaaCCCCttcacacttat
cttcaaactcacacacaatactcactcactagtttcatattcatattttttgaaacatgtctGGtgtttGGaaaatc
aagaatGGagtagtgaGGctagttgagaaCCtcGGtgactttcacGGtgcgacGGGtcgtcgtaaagtgcttgtgca
CCtttctagtaatgaagtaataacatcatatgcagtacttgaaaGGaaactgtactctcttGGatGGGagaGGtact
atgatgaCCCtgaCCttcttcagtaCCataaaagatcaactgttcatcttatttctctaCCaaacgacttcaacaaC
CtcaGGtCCatgcacatgtatgatattgttgttaagaatcgtaatgagtttgctgttaGGGatatgtagtattacta
ataatcattagttgatttgagatttttctcaaattaattaatgttgtttaatttaaattaGGttgtttcttctttta
acttaaGGtttGGtttgtgtaatttaGGtcaaaGGGGGGtgttttagtttcttttGGGtgaGGaagctaattattac
ttgttgtaatGGtgtgtaagagtgaagtttatGGcaataaaacttGGtttcgcttcgaaacttttatctatatactt
aaataaatttgtactatcaaatacttaaatttttagtcatatatatatttaaaagtcttctttatttacttaaattt
tgtatcaagtcaaaCCagattatatttttatcattaagCCaacgatgataGGtGGatatgtgattgatatatttttt
tttatGGaaatatcttttcttttctctttttttttttGGtcttattttgaataaagacaaaatGGtattttCCCatt
tatttcatcaagaagtctttgactataaattcaaaGGctttaCCtcaaattcgaattcttcactgttttaaaaaaat
aaagtaagatgtcaagaatatatatatatatatatatatatatatatatatatatatatatatatatatatatatat
atatatatatatatatatatcttttGGGaaatttaattaaattattatgaagcaaataaaGGGtaaaagaacaaata
aataaatgcaatcaaataaatgaagaGGtaatatGGacttGGGcttttcaGGctgctaatttGGGttctGGCCCtat
ttaaaCCtttgaaaacttttgtatacaacaagtgtatattgatatatacagatcgtttctaagCCtttttCCtgtat
atcaactgtatacagCCtgttctaatgCCtCCaaCCtgtatcttcatttttgtcaacatatatgttCCtgaacatat
agatcgctgtatacatattgtatacattatgtatacaactcatttcttGGGcttttgaattatttCCaat
Solyc01g066950 locus (ORF): SEQ ID NO: 2 (5′→3′)

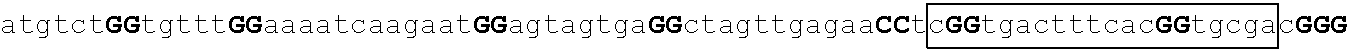

tcgtcgtaaagtgcttgtgcaCCtttctagtaatgaagtaataacatcatatgcagtacttgaaaGGaaactgtact
ctcttGGatGGGagaGGtactatgatgaCCCtgaCCttcttcagtaCCataaaagatcaactgttcatcttatttct
ctaCCaaacgacttcaacaaCCtcaGGtCCatgcacatgtatgatattgttgttaagaatcgtaatgagtttgctgt
taGGGatatgtag
Solyc01g066950 locus (encoded amino acid sequence): SEQ ID NO: 3
MSGVWKIKNGVVRLVENLGDFHGATGRRKVLVHLSSNEVITSYAVLERKLYSLGWERYYDDPDLLQYHKR
STVHLISLPNDENNLRSMHMYDIVVKNRNEFAVRDM
Solyc01g066950 locus guide sequence #2: SEQ ID NO: 5 (5′→3′)
cggtgacttccacggtgcga
Solyc01g066970 locus: SEQ ID NO: 6 (5′→3′)
tttctctgtcttgtcttgaaaaaagaatgttttttttttttttataattctttactttcaattcttttacatgtgat
ctttagaagacaagattaaataacattttgatactttctatatattttaattataaaatcacaagattcagaagtct
tgtttattttttaaaacttcatgtcaaactaaaactagataaacaaattGGaacagacactatCCCattgaaatttt
CCtattgaaaaatgtCCagtGGctatactcacactaatgtttaaattacacaacaaaattaaaaaaaaaactcttGG
tattttagtgagaatttgtttctcaCCatacgtttttattgaCCtagttaaataGGaaatGGGtGGGaatatcacgt
atcataacacaaatttctcattgatttGGagtaattttttttttttaaaaaaaaattgttattagacattaattaaG
GattaaaagaaacatcatcaacatgagatGGGacaaattaatcttCCCCgaaatatcttttaatttatttaattctt
CCtttttgtgaaGGGctgatcaagcaatGGatataagaatagaagattgttcttagcactaaaaaaattaaagaatt
atgcttGGaaCCCattaaCCaaaagaattaGGttcatcttatgagcataagatcattaattagtgattgtttaGGag
aagattctaatttcagtaGGGcaaattaGGGcatcttgtGGCCatttaaatattctCCCtttctttttctttaatct
taataaacgtacgataagttagtatatttctaaatCCtataagcagCCacattCCaaaatCCtaCCtattatcaatt
ttattaaataagaaaaaagattactttttgCCaCCttatgtatttttttattacacactacatagaaaCCCCtataa
aaaCCCactcacacttatgttcaaactcacacacaatactcacttactattttcatattcatatattttttgaaaca
tgtctGGtgtttGGGtattcaagaatGGagtagtgaGGctagttgagaaCCCCGGtgacttCCacGGtgcgacGGGt
cgtcgtaaagtgcttgtgcaCCtttctagtaatgaagtaataacatcatatgcagtacttgaaaGGaaactgtactc
tcttGGatGGGagaGGtactatgatgaCCCtgaCCttcttcaattCCataaaagatcaactgttcatcttatttctc
taCCaaaGGacttcaacaaCCtcaagtCCatgcacatgtatgatattgttgttaagaatcgtaatgagtttacagtt
aGGGatatgtagtactactaattaataattagttgatttgagatatttttctcaaattaattaatgttgtttgattt
aaattaGGttgtttcttgttttaacttaatgtttGGtttgtgtaatttaGGtttaaGGGGGGtgttttagtttcttt
tGGGtgaGGaagctaattattacttgtaattgtgtgtaagagtgaagttttatGGcaataaaaaaacttGGtttGGc
ttgaaaattttatctatatactgaaataaattcttactatcaaatacttcaattttgagtctctcacacacgcgcat
atatatatatatatatatatatatatatatatatatatatatatatatacttCCCCgtttaaaaaagaataatcttc
tttCCtttttagttttttttttCCCCgtttaaaaaagaataatcttctttCCtttttagtttttttttttatataaa
agaatgacttttttttGGttacattttaactttagctttCCacgtaattaatttagcgctacttttcaattacaaat
tctgctttattaaatctGGttaatgatatttgaaaaattttaatttgtgaGGcaaattttaGGttaagatactcgaa
gagtttttcttaagatagttcacataaGGttttgcaaaagttGGGagaaattgttatatttgaactagCCCtatttc
tagcttatgtatgaatttgaaataataataatttaactatcaaattaattatgtatacaagataactcgaataattt
gtatatagattatctctaacagatgCCttgtaGGGtattaaatttgCCtgcaaGGctttttCCagtttgttttctgt
ataataatatgtagcatGGcatctattCCcttttttaataaatatctattcataatcagacgtctaaaattcgaata
cttttcttgataatatcgtcttactCCttaattagtaagttgtgttgtcattaaatat
Solyc01g066970 locus (ORF): SEQ ID NO: 7 (5′→3′)

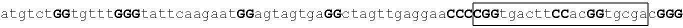

tcgtcgtaaagtgcttgtgcaCCtttctagtaatgaagtaataacatcatatgcagtacttgaaaGGaaactgtact

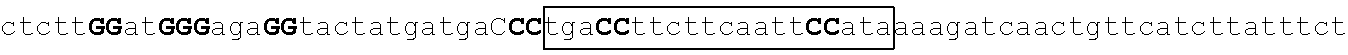

ctaCCaaaGGacttcaacaaCCtcaagtCCatgcacatgtatgatattgttgttaagaatcgtaatgagtttacagt
taGGGatatgtag
Solyc01g066970 locus (encoded amino acid sequence): SEQ ID NO: 8
MSGVWVFKNGVVRLVENPGDFHGATGRRKVLVHLSSNEVITSYAVLERKLYSLGWERYYDDPDLLQFHKRSTVHLIS
LPKDENNLKSMHMYDIVVKNRNEFTVRDM
Solyc01g066970 locus guide sequence #1: SEQ ID NO: 9 (5′→3′)
tatggaattgaagaaggtca
Solyc01g066970 locus guide sequence #2: SEQ ID NO: 10 (5′→3′)
cggtgacttccacggtgcga
Solyc06g005530 locus: SEQ ID NO: 11 (5′→3′)
tttattagagatgtcatttgataatatttttattatttcttcttcttattattttttGGttaagttatcttcttttc
ttttttttctctctttctatatttttaCCatttaacgaaaataaataaataaattacttttatattttcaaaatgac
atagttgaaCCttatcaaGGtgtttaaaatataaaaagtctacttgaaatgtttaaaagtgaaagtttatgttactt
ttaaGGatttgacgatgaattttagtatCCtaCCatatatttgaaacagcttgtctcatcattgtGGtacaaatgat
aagataaatattttttttttgttttttgtttttcatCCGGtgttcgatatcaacaatGGaaCCCaataatattcaga
ttcttacgaaacgtCCtacatctgaGGGtaaaatactCCttaacagagatgactCCatagttagagaGGataaataa
tctcaagatcactaaattaatatCCCtaaCCaaatacaagataaaatgtgtCCCacaattataactCCCtatatCCC
actttatacgacacttttcagatttcgacattcaaacaattctattttttaCCgtaaaaaatatcatatcttgaatt
atcaatacaaatatataatttcatttaatttttaaaaaagattCCattagtaaattttcaattaagcttaaactaaa
cagaaaaaaaatatctcttatCCatcgtaCCaaacgacaCCagaacataaaaattaaaaaaCCtagaaagtaaatga
actagtatCCCaaaaaGGttaatagtagtCCagtcattcaaaagatcagtgatcacatgatgtactagcaaaCCtac
atacacagtGGaatatatctactgctCCataagaaattatttcatcatttctctaagagttatgaattattttatta
ttatttttctttctCCatctCCatatattgttGGagttGGaaactaatataaagtaaattaaaCCattatattataa
tgtctGGcgtatGGatatttgacaagaaaGGtgttgCCCatttgatcaaaaatCCtactcgtgaatCCttcgagcta

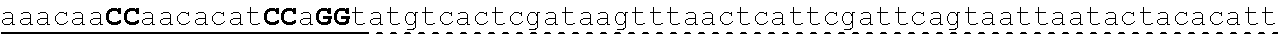

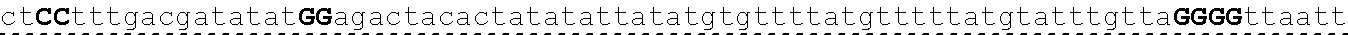

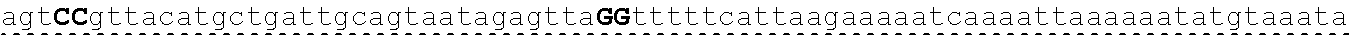

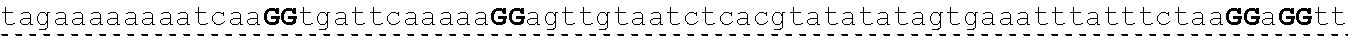

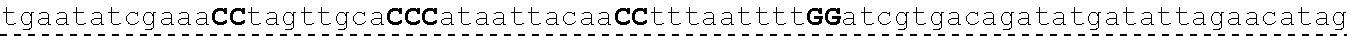

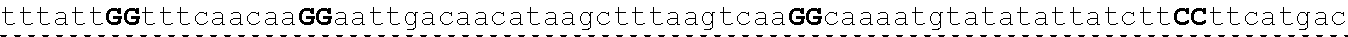

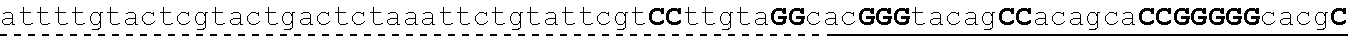

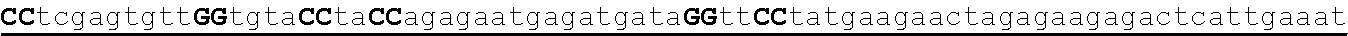

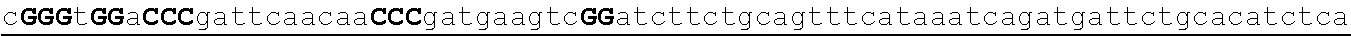

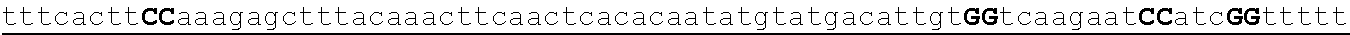

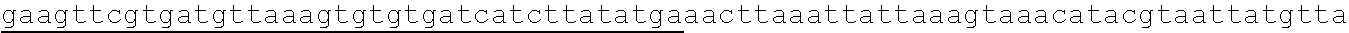

tttctCCtctttcgtatctgagcttgattctttttcattagCCaaatacgtGGatttatatgttttttcgctctttG
GttGGtGGagagatttGGaaCCtagctagcatatctcgtgCCtattctgataacatattgaattgtatacatgatcg
tttcatctaaaaattaagcatttaaaaatcacatttttaattacttaactaattatatagttatattatgtgttaac
acataCCttgcatatatgagtttgattcttcttcatgaCCagaaaatgtcaaatatatttttgctttttgagCCGGa
actcttacatgctCCatttgataacaagttGGaatgtgtGGttatcttatataaaaacaacacaagttgttatgaaa
agcatatttataattattaatctaattatattatgttttaacacagttttttatatgtgaatttgattctttttcat
tGGGcaaacatgtgtgaaacaatttatttttttatagactaGGatgatagagaaatttgaacttaaaatctctcatg
tgttcgaataacatattataaagtatgctatcattcatttaaaatttaacttattagaaaataatacactttttttt
acttaattataatgtgactcttcgttttGGCCataataaaagtctatttgaattgatttttgacttttgattttcaa
gtcaatgtttgaattatttttgatgtttttagcttaaagcaaatGGtttgtgcgtCCaaaaaatatttgaaactatt
ttaacttaaaatcacttaaaacaagtcgatCCatgtaacatgcaagttttgaCCtaattagaaGGtttgaaattata
CCtagctagagctatctatttcttttattatcaatttttttaatatatcatagttctatattaatatttttttgctt
tctcgata
Solyc06g005530 locus (ORF): SEQ ID NO: 12 (5′→3′)
atgtctGGcgtatGGatatttgacaagaaaGGtgttgCCCatttgatcaaaaatCCtactcgtgaatCCttcgagct
aaaacaaCCaacacatCCaGGtatatgtcactcgatataagtttaactcattcgattcagtaattaatactacacat
tctCCtttgacgatatatGGagactacactatatattatatgtgttttatgtttttatgtatttgttaGGGGttaat
tagtCCgttacatgctgattgcagtaatagagttaGGtttttcattaagaaaaatcaaaattaaaaaatatgtaaat
atagaaaaaaaatcaaGGtgattcaaaaaGGagttgtaatctcacgtatatatagtgaaatttatttctaaGGaGGt
ttgaatatcgaaaCCtagttgcaCCCataattacaaCCtttaattttGGatcgtgacagatatgatattagaacata
gtttattGGtttcaacaaGGaattgacaacataagctttaagtcaaGGcaaaatgtatatattatcttCCttcatga
cattttgtactcgtactgactctaaattctgtattcgtCCttgtaGGcacGGGtacagCCacagcaCCGGGGGcacg
CCCtcgagtgttGGtgtaCCtaCCagagaatgagatgataGGttCCtatgaagaactagagaagagactcattgaaa
tcGGGtGGaCCCgattcaacaaCCCgatgaagtcGGatcttctgcagtttcataaatcagatgattctgcacatctc
atttcacttCCaaagagctttacaaacttcaactcacacaatatgtatgacattgtGGtcaagaatCCatcGGtttt
tgaagttcgtgatgttaaagtgtgtgatcatcttatatga
Solyc06g005530 locus (encoded amino acid sequence): SEQ ID NO: 13
MSGVWIFDKKGVAHLIKNPTRESFELKQPTHPGTGTATAPGARPRVLVYLPENEMIGSYEELEKRLIEIGWTRENNP
MKSDLLQFHKSDDSAHLISLPKSFTNENSHNMYDIVVKNPSVFEVRDVKVCDHLI
Solyc06g005530 locus guide sequence #1: SEQ ID NO: 14 (5′→3′)
agagactcattgaaatcggg
Solyc06g005530 locus guide sequence #2: SEQ ID NO: 15 (5′→3′)
Agaagagactcattgaaatc
Solyc06g005530 locus guide sequence #3: SEQ ID NO: 76 (5′→3′)
Gagaagagactcattgaaat
Solyc06g005530 locus guide sequence #4: SEQ ID NO: 77 (5′→3′)
Gggggcacgccctcgagtgt
Solyc06g005530 locus guide sequence #5: SEQ ID NO: 78 (5′→3′)
Ggtaggtacaccaacactcg
Solyc06g005530 locus guide sequence #6: SEQ ID NO: 79 (5′→3′)
Aacactcgagggcgtgcccc
Solyc06g005530 locus guide sequence #7: SEQ ID NO: 80 (5′→3′)
Gggtacagccacagcaccgg
Solyc06g005530 locus guide sequence #8: SEQ ID NO: 81 (5′→3′)
cgggtacagccacagcaccg
Solyc06g005530 locus guide sequence #9: SEQ ID NO: 82 (5′→3′)
Acgggtacagccacagcacc
Solyc06g005530 locus guide sequence #10: SEQ ID NO: 83 (5′→3′)
Cacgggtacagccacagcac
Solyc06g005530 locus guide sequence #11: SEQ ID NO: 84 (5′→3′)
Agggcgtgcccccggtgctg
Solyc06g005530 locus guide sequence #12: SEQ ID NO: 85 (5′→3′)
Tgtattcgtccttgtaggca
Solyc06g005530 locus guide sequence #13: SEQ ID NO: 86 (5′→3′)
Aattctgtattcgtccttgt
Solyc06g005530 locus guide sequence #14: SEQ ID NO: 87 (5′→3′)
Tggctgtacccgtgcctaca
Solyc06g005530 locus guide sequence #15: SEQ ID NO: 88 (5′→3′)
Acgagtacaaaatgtcatga
Solyc06g005530 locus guide sequence #16: SEQ ID NO: 89 (5′→3′)
Acaacataagctttaagtca
Solyc06g005530 locus guide sequence #17: SEQ ID NO: 90 (5′→3′)
ttgtaattatgggtgcaact
Solyc06g005530 locus guide sequence #18: SEQ ID NO: 91 (5′→3′)
Agtaggatttttgatcaaat
Solyc06g005530 locus guide sequence #19: SEQ ID NO: 92 (5′→3′)
aaccattatattataatgtc
Solyc12g099610 locus: SEQ ID NO: 16 (5′→3′)
aagttttgaattctttaGGttgctttttctttaatttttttcttcttctcatatcatgaatcttatCCatttcaata
tttCCaCCaaacatGGGacatGGacatctctatgagttcatcttcttgcttCCaatgcattatctGGtgtttgatat
tcgtattgagcttCCactaattcagattcatgCCgcataaagtctatttaaaagaaaaatatttctatcaaaattgt
tttcatactctaGGGtcgagcaaaGGGattcatgaCCaatgatatctacGGGaatattaaagaatcttgataaagaa
cacttctCCttgtCCgagCCtttgacaaaaatcatttttGGtaGGattgcttCCCCaCCtttcagtcttatgtagaa
tttgaattagttgagattcactatgaatatcgaataaataacaaaaaaaaaaaGGagtaatgaatctttCCaaatat
agaatatattatgattaaatgcatgcatGGGaagcaaaaagatgaacttatGGagatgtgtcatgtCCCatatattt
gatGGaaatattGGGttGGataagattcatgatgaaaaaaaaaagcGGtgacataaatctgaattagtcGGaactCC
aaatagtttaatttgtttttgaaaaataaCCttcttttacttgCCCtttCCttttttatctcttcaaaaaataaaaa
taaaacttcttaCCacaatttatactatatattacttattaaGGGGaatcttgatgcaataacataacacagttatc
tttatcagattcgaaCCgtagaagcagctacaaatatttgtaataaGGaaGGctatttacatcacacatgtatttat
acgtatatGGacttatttatttatttatatatatatatatatatatatgcatatcacaCCatgcattaaCCCtataa
aaCCCacacattatattctttttcaacaacaCCatcttttacatatattcaacttCCCCtCCCtctatCCCtcatca
tgtcaGGtgtttGGattttcaaaaacGGcgtcgtCCGGctagaaaCCCCCGGtgactgCCacgtcagctCCacgaCC
GGtcatcGGaaagttctagtacatgttCCtagtaaagaagtcattacatgttatgcaaatcttgaaaaaaagcttta
tagtcttGGatGGGaaaGGtattatgatgatCCacaacttcttcaataCCacaaaagatCCacaattcatcttattt
CCCtCCCaattgattttaataGGtttaaatCCattcatatgtatgatattgttgttaaaaatcgaaatgaatttgaa
gttagagatatgtaaagttactaactttctttacgtGGatataagaaatgtgaaatttGGagaaacttatgtgtttt
cgagttgatagtgatatgtttGGagattGGagttgtgtttgaacatGGatacgaacGGaattgtttttgaatttttg
aaagtgaaaattgctttttattgtttttgaacttaaaattgttatgtGGctaacaaaataaaatcaatcaacaaaca
agtcgttgtagtatagtGGtaagtattCCCgCCtgtcacgcGGGtgaCCCGGGttcgatCCCCGGcaacGGcgttaa
ttttttttatgtttctacacataCCatatatctagttatatcttacgacaagcacaaatacattatgctctcgcaac
atacaatgtatctagttatatatcttacgagaagcacaaatacattatgctctcgcaacatacaatatatCCagttg
tgtcttacgacaagtactCCaaaaCCCaCCaacgctcgagaaatgCCttgttatGGtgtaagaaacatcagcttcag
tatgttaagactgataacaaaGGagttacttcacaagttctttttcaacaagtaatttacatagagtttGGatgttg
tgttctGGacaacaagaaaaaatgaatgtagttagtctaaGGctatgttgcttGGactctCCaaaagatgctacaCC
CgtgtcGGGtCCtCCaaaaatgcactacttttgaaGGatcagacatgcacgtgtcgCCatatttcaagagCCCgagc
aacataGGttcaaGGaactcatatgatataGGctaatgtcacgaactcactttcttctttgtcgtgctCCaaatgtt
tcagctctgaaCCtatacattCCgCCatCCaatatatctCCtcagtCCgcGGGtgagacttgtcatCCgat
Solyc12g099610 locus (ORF): SEQ ID NO: 17 (5′→3′)
atgtcaGGtgtttGGattttcaaaaacGGcgtcgtCCGGctagaaaCCCCCGGtgactgCCacgtcagctCCacgaC
CGGtcatcGGaaagttctagtacatgttCCtagtaaagaagtcattacatgttatgcaaatcttgaaaaaaagcttt
atagtcttGGatGGGaaaGGtattatgatgatCCacaacttcttcaataCCacaaaagatCCacaattcatcttatt
tCCCtCCCaattgattttaataGGtttaaatCCattcatatgtatgatattgttgttaaaaatcgaaatgaatttga
agttagagatatgtaa
Solyc12g099610 locus (encoded amino acid sequence): SEQ ID NO: 18
MSGVWIFKNGVVRLETPGDCHVSSTTGHRKVLVHVPSKEVITCYANLEKKLYSLGWERYYDDPQLLQYHKRSTIHLI
SLPIDENRFKSIHMYDIVVKNRNEFEVRDM
Solyc12g099610 locus guide sequence #1: SEQ ID NO: 19 (5′→3′)
ccctcatcatgtcaggtgtt
Solyc12g099610 locus guide sequence #2: SEQ ID NO: 20 (5′→3′)
ttttcaaaaacggcgtcgtc
Solyc12g099610 locus guide sequence #2: SEQ ID NO: 93 (5′→3′)
Agtcaccgggggtttctagc
Solyc12g099610 locus guide sequence #2: SEQ ID NO: 94 (5′→3′)
Gtcgtccggctagaaacccc
Solyc12g099610 locus guide sequence #2: SEQ ID NO: 95 (5′→3′)
Agctgacgtggcagtcaccg
Solyc12g099610 locus guide sequence #2: SEQ ID NO: 96 (5′→3′)
Gagctgacgtggcagtcacc
Solyc12g099610 locus guide sequence #2: SEQ ID NO: 97 (5′→3′)
Gaccggtcgtggagctgacg
Solyc12g099610 locus guide sequence #2: SEQ ID NO: 98 (5′→3′)
Aactttccgatgaccggtcg
Solyc12g099610 locus guide sequence #2: SEQ ID NO: 99 (5′→3′)
Gatgaattgtggatcttttg
Solyc12g099610 locus guide sequence #2: SEQ ID NO: 100 (5′→3′)
Gagggaaataagatgaattg
Solyc12g099610 locus guide sequence #2: SEQ ID NO: 101 (5′→3′)
Ccctoccaattgattttaat
Solyc01g066980 locus: SEQ ID NO: 21 (5′→3′) (br locus)
atgtctGGagtttGGGtattcaagaatGGtgttgtCCgtctagtGGagaactCCgattgCCacGGGGcgaacGGact
CCgaaaagttcttgtacatcttCCtagtaatgaagtcatcacatcatatgcagtacttgaaaGGaaactgtactctc
ttGGatGGGagaGGtactatgatgaaCCtgaacttcttcaataCCacaaaagatcaaCCgttcatcttatttctcta
CCaaaGGatttcaacaGGttcaaatCCatgcatatgttcgatatcgtcgtcaagaatcgcaatgaatttgaGGttag
agatatg
Solyc01g066980 locus (amino acid sequence): SEQ ID NO: 22
MSGVWVFKNGVVRLVENSDCHGANGLRKVLVHLPSNEVITSYAVLERKLYSLGWERYYDEPELLQYHKRSTVHLISL
PKDFNRFKSMHMFDIVVKNRNEFEVRDM
Solyc01g066980 locus: SEQ ID NO: 102
catctcatcataaactacaaacacatacaaaaaacattctcattcaCCtttCCtctacaaaaaacataacaacatct
tcaacaatcatgtctGGagtttGGGtattcaagaatGGtgttgtCCgtctagtGGagaactCCgattgCCacGGGGc
gaacGGactCCgaaaagttcttgtacatcttCCtagtaatgaagtcatcacatcatatgcagtacttgaaaGGaaac
tgtactctcttGGatGGGagaGGtactatgatgaaCCtgaacttcttcaataCCacaaaagatcaaCCgttcatctt
atttctctaCCaaaGGatttcaacaGGttcaaatCCatgcatatgttcgatatcgtcgtcaagaatcgcaatgaatt
tgaGGttagagatatgtaaacaaaatatGGGGgaaaaaaGGGaaGGagttgatcatttgaatgtgtttttttttctt
ttttttgcttttttttGGtcaagtgtgttgtaattaagtttctatcgtttaatttgtgatttgtttcacaatgttgc
taaGGttgtaatttGGaaagttgtaagaGGGGaaatgttgtatattattacaagtgaatgtgttttattatatgata
tatatatatataagag

TABLE 2
(part A). Brachytic loci homologs, amino acid sequence
alignment part 1 (sequences are continued in parts B-F).

| | |
|---|---|
| Niben101Scf00012g00011.1 | MSGVWIFDKKGVARLITNPT |
| Peaxi162Scf00056g00139.1 | MSGVWIFDKKGVAHLIKNPT |
| Peinf101Scf01105g01005.1 | MSGVWIFDKKGVAHLIKNPT |
| Capana06g002723 | MSGVWIFDKKGVAHLIKNPT |
| Capang06g002516 | MSGVWIFDKKGVAHLIKNPT |
| Capang05g001509 | GTG |
| SMEL 006g247790.1.01 | MSGVWIFDKKGVAHLIKNPT |
| PGSC0003DMP400007817 | MSGVWIFDKKGVAHLIKNPT |
| Sopen06g001510.1 | MSGVWIFDKKGVAHLIKNPT |
| Solyc06g005530.2.1 | MSGVWIFDKKGVAHLIKNPT |
| Niben101Scf05107g01003.1 | MSGVWLSKNTGVIRLLENQTE |
| Peinf101Scf02016g05027.1 | MSGVWVF-KNGVERLVENPG |
| Peaxi162Scf00078g00059.1 | MSGVWVF-KNGVFRLVENPG |
| SMEL 012g387130.1.01 | MSGVWVF-KNGVFRLVENG |
| Capana10g001758 | MSGVWVF-KNGVERLVENG |
| CA05g11610 | LIFEKEHTHTHTSEVEMSGVWVF-KNGVERLVENG |
| Niben101Scf05041g04001.1 | MSGVWVF-KNGVERLVENP |
| Niben101Scf02182g12004.1 | MSGVWVF-KNGVERLVENP |
| Sopen01g028590.1 | MPGVWEI-KNGVVRLVEKPG |
| Niben101Scf13863g00010.1 | MSGVWVF-KNGVLRLVENPG |
| SMEL 001g140830.1.01 | MSGVWVF-KNGVVRLVENTG |
| Capana01g003223 | MSGVWVF-KNGVVRLVEN-G |
| Solyc01g066980.3.1 | SHHKLQTHTKNILIHLSSTKNITTSSTIMSGVWVF-KNGVVRLVENS |
| Sopen01g028640.1 | MSGVWVF-KNGVVRLVENS |
| Sopim01g066980.0.1 | MSGVWVF-KNGVVRLVENS |
| PGSC0003DMP400020089 | MSGVWVF-KNGVVRLVENS |
| Niben101Scf10524g05008.1 | MSGVWVF-KNGVVRLE |
| Peaxi162Scf00534g00012.1 | MSGVWVF-KNGVVRLVENPG |
| Peinf101Scf01113g00005.1 | MSGVWVF-KNGVVRLVENPG |
| Peaxi162Scf00086g00036.1 | MSGVWVF-KNGVLRLVENPGDNYHG |
| Peinf101Scf00973g06042.1 | MSGVWVF-KNGVLRLVENPGDNYHG |
| Capana01g003222 | MSGVWVF-KNGVVRLVENPG |
| Peaxi162Scf00534g00005.1 | MSGVWVF-KNGVVRLVENPG |
| Peinf101Scf01113g00004.1 | MSGVWVF-KNGVVRLVENPG |
| SMEL 001g140850.1.01 | MSGVWVF-KNGVVRLVENPG |
| Niben101Scf02626g03001.1 | MSGVWVF-KNGVVRLVENPG |
| Niben101Scf10524g05006.1 | MSGVWVF-KNGVVRLVENPG |
| Solyc01g066970.2.1 | MSGVWVF-KNGVVRLVENPG |
| Sopen01g028630.1 | MSGVWVF-KNGVVRLVENPG |
| PGSC0003DMP400020088 | MSGVWVF-KNGVVRLVENAG |
| Sopen01g028610.1 | MSGVWKI-KNGVVRLVENLG |
| Solyc01g066950.1.1 | MSGVWKI-KNGVVRLVENLG |
| Capana12g000135 | MSGVWTF-KNGVVRL-ENRG |
| Capang12g000108 | VS |
| SMEL 005g240480.1.01 | MSGVWVF-KNGVVRL-ENPG |
| Solyc12g099610.1.1 | MSGVWIF-KNGVVRL-ETPG |
| PGSC0003DMP400008206 | MSGVWIF-KNGVVRL-ENPG |

(part B). Brachytic loci homologs, amino acid sequence alignment part 2.

| | |
|---|---|
| Niben101Scf00012g00011.1 | |
| Peaxi162Scf00056g00139.1 | |
| Peinf101Scf01105g01005.1 | |
| Capana06g002723 | |
| Capang06g002516 | |
| Capang05g001509 | |
| SMEL 006g247790.1.01 | |
| PGSC0003DMP400007817 | |
| Sopen06g001510.1 | |
| Solyc06g005530.2.1 | |
| Niben101Scf05107g01003.1 | |
| Peinf101Scf02016g05027.1 | |
| Peaxi162Scf00078g00059.1 | |
| SMEL_012g387130.1.01 | |
| Capana10g001758 | |
| CA05g11610 | |
| Niben101Scf05041g04001.1 | |
| Niben101Scf02182g12004.1 | |
| Sopen01g028590.1 | |
| Niben101Scf13863g00010.1 | |
| SMEL_001g140830.1.01 | |
| Capana01g003223 | |
| Solyc01g066980.3.1 | |
| Sopen01g028640.1 | |
| Sopim01g066980.0.1 | |
| PGSC0003DMP400020089 | |
| Niben101Scf10524g05008.1 | |
| Peaxi162Scf00534g00012.1 | |
| Peinf101Scf01113g00005.1 | |
| Peaxi162Scf00086g00036.1 | SRKVLVHVPSDEVITSYAILERKLYNLGWERYYDDPNLLQYHKRSTVHLISLPR |
| Peinf101Scf00973g06042.1 | SRKVLVHVPSNEVVTSYAILERKLYNLGWERYYDDPNLLQYHKRSTVHLISLPR |
| Capana01g003222 | |
| Peaxi162Scf00534g00005.1 | |
| Peinf101Scf01113g00004.1 | |
| SMEL 001g140850.1.01 | |
| Niben101Scf02626g03001.1 | |
| Niben101Scf10524g05006.1 | |
| Solyc01g066970.2.1 | |
| Sopen01g028630.1 | |
| PGSC0003DMP400020088 | |
| Sopen01g028610.1 | |
| Solyc01g066950.1.1 | |
| Capana12g000135 | |
| Capang12g000108 | |
| SMEL 005g240480.1.01 | |
| Solyc12g099610.1.1 | |
| PGSC0003DMP400008206 | |

(part C). Brachytic loci homologs, amino acid sequence alignment part 3.

| | |
|---|---|
| Niben101Scf00012g00011.1 | RESFDLMQPTSSGTGT--APGARPKVLVYLPENQ |
| Peaxi162Scf00056g00139.1 | RESFELKEPTYPGTGTATAPGARPKVLVYLPENE |
| Peinf101Scf01105g01005.1 | RESFELNEPTYPGTGTATAPGARPKALVYLPENE |
| Capana06g002723 | RESFELKOPAYPGTGTATAPGARPRVLVYLPENE |
| Capang06g002516 | RESFELKQPAYPGTGTATAPGARPRVLVYLPENE |
| Capang05g001509 | TAT--APGARPRVLVYLPENE |
| SMEL_006g247790.1.01 | RESFELKQSTYPGTGTATAPGARPRVLVYLPENE |
| PGSC0003DMP400007817 | RESFELKQPTYPGTGTVTAPGARPRVLVYLPENE |
| Sopen06g001510.1 | RES FELKQPTHPGTGTATAPGARPRVLVYLPENE |
| Solyc06g005530.2.1 | RESFELKQPTHPGTGTATAPGARPRVLVYLPENE |
| Niben101Scf05107g01003.1 | EEQ--SIGRKRKVLVHLPTQE |
| Peinf101Scf02016g05027.1 | AEQ---AQRRRKVLVHLPTGQ |
| Peaxi162Scf00078g00059.1 | AEQ---AQRRRKVLVHLPTGQ |
| SMEL 012g387130.1.01 | SGD--QAQRRRKVLIHLPSGQ |
| Capana10g001758 | SGD--QAQRRRKVLLHLPSGQ |
| CA05g11610 | SGD--QAQRRRKVLLHLPSGQ |
| Niben101Scf05041g04001.1 | SSE--QGQRRRKVLVHLPTGQ |
| Niben101Scf02182g12004.1 | SSE--QGQRRRKVLLHLPTGQ |
| Sopen01g028590.1 | DSH--GATVRNKVLVHLSSNE |
| Niben101Scf13863g00010.1 | DHF----QGCRKVLVHIPTNE |
| SMEL_001g140830.1.01 | DCQ--GANGGRKVLVHVPSDE |
| Capana01g003223 | DCQ--GVNGCRKVLVHLASGE |
| Solyc01g066980.3.1 | DCH--GANGLRKVLVHLPSNE |
| Sopen01g028640.1 | DCH--GANGLRKVLVHLPSNE |
| Sopim01g066980.0.1 | DCH--GANGLRKVLVHLPSNE |
| PGSC0003DMP400020089 | DCH--GANGLRKVLVHLPSDE |
| Niben101Scf10524g05008.1 | DCQ--GSSGRRKVLVHVPSNE |
| Peaxi162Scf00534g00012.1 | DCQ--GSSGRRKVLVHVPTNE |
| Peinf101Scf01113g00005.1 | DCQ--GSSGRRKVLVHVPTNE |
| Peaxi162Scf00086g00036.1 | DFSKLKTMHMYDIVVKNRNEFESNGVVRLENPSDYH--GSAGRRKVLVHAASNE |
| Peinf101Scf00973g06042.1 | DFSKFKTMHMYDIVVKNRNEFESNGVVRLENPGDYH--GSSGRRKVLVHATSNE |
| Capana01g003222 | DCH--GATGRRKVLVHLASNE |
| Peaxi162Scf00534g00005.1 | DFH--GSSGRRKVLVHVPSNE |
| Peinf101Scf01113g00004.1 | DFH--GSTGRRKVLVHVPSNE |
| SMEL_001g140850.1.01 | DFH--GSTGRRKVLVHLPSNE |
| Niben101Scf02626g03001.1 | DCH--GATGRRKVLVHLSSNE |
| Niben101Scf10524g05006.1 | DCH--GATGRRKVLVHLSSNE |
| Solyc01g066970.2.1 | DFH--GATGRRKVLVHLSSNE |
| Sopen01g028630.1 | DFH--GATGRRKVLVHLSSNE |
| PGSC0003DMP400020088 | DFH--GATGRRKVLVHLSSNE |
| Sopen01g028610.1 | DFQ--GATGRRKVLVHLSSNE |
| Solyc01g066950.1.1 | DFH--GATGRRKVLVHLSSNE |
| Capana12g000135 | DCHVSATTGRRKVLVHVASDE |
| Capang12g000108 | ATAGRRKVLVHVASDE |
| SMIEL 005g240480.1.01 | DCHVSSTTSRRKVLVHVPSNE |
| Solyc12g099610.1.1 | DCHVSSTTGHRKVLVHVPSKE |
| PGSC0003DMP400008206 | DCHVSSTTGHRKVLVHVPSNE |

(part D). Brachytic loci homologs, amino acid sequence alignment part 4.

| | |
|---|---|
| Niben101Scf00012g00011.1 | VISSYADLEKILIELGWSRYNNPIRLDFMQFHKSDDSAHL-ISLPKEFTNFKSL |
| Peaxi162Scf00056g00139.1 | VISSYDELEKILVELGWSRYNNPTRSDLLQFHKSDDSAHL-ISLPISFTNFKPL |
| Peinf101Scf01105g01005.1 | VISSYDELEKILIELGWSRYNSPTRSDLLQFHKSNDSGHL-ISLPISFTNFKPL |
| Capana06g002723 | MISSYEELERRLIELGWTRENNPMRSDLLQFHKSDDSAHL-ISLPKSFTDFKSL |
| Capang06g002516 | MISSYEELERRLIELGWTRENNPMRSDLLOFHKSDDSAHL-ISLPKSFTNEKSL |
| Capang05g001509 | MISSYEELERRLIELGWTRENNPMRSDLLQFHKSDDSAHL-ISLPKSFTNFKSL |
| SMEL 006g247790.1.01 | IISSYEELERRLIELGWTRENNPMKSDLLQFHKSDDSAHL-ISLPKSFTNFKSL |
| PGSC0003DMP400007817 | MISSYEELEKRLIELGWTRENNPMKSDLLQFHKSDDSAHL-ISLPKSFTNFKSH |
| Sopen06g001510.1 | MIGSYEELEKRLIEIGWTRENNPMKSDLLQFHKSDDSAHL-ISLPKSFTNFNSH |
| Solyc06g005530.2.1 | MIGSYEELEKRLIEIGWTRENNPMKSDLLQFHKSDDSAHL-ISLPKSFTNENSH |
| Niben101Scf05107g01003.1 | IVSSYNSLDKILTDLGWEKYDCGDDPHFYQFHKRT-PIHLSLSLPNDFAKFNTV |
| Peinf101Scf02016g05027.1 | MVSSYCSLERILNGLGWERV |
| Peaxi162Scf00078g00059.1 | MVSSYCSLERILNGLGWERYYGG-DPELFQFHKHS-SIDL-ISLPKDFSKENSI |
| SMEL_012g387130.1.01 | VVSSYCSLERILNDLGWERYYEG-DAELFQFHKHS-SIDL-ISLPMDFTKENSI |
| Capana10g001758 | VVSSYCSLERILNGLGWERYYGG-DTELFQFHKHS-SIDL-ISLPKDFAKENSI |
| CA05g11610 | VVSSYCSLERILNGLGWERYYGG-DTELFQFHKHS-SIDL-ISLPKDFAKFNSI |
| Niben101Scf05041g04001.1 | VVSSYCSLERILKGLGWERYYGG-DPELFQFHKHS-SIDL-ISLPKEFAKENSI |
| Niben101Scf02182g12004.1 | VVSSYCSLERILNGLGWERYYGG-DPELFQFHKHS-SIDL-ISLPKDFAKFNSI |
| Sopen01g028590.1 | VITSYASLERILISIGWERYYDG-DPDLLQYHKRS-TVHI-ISLPKDFKNFKFP |
| Niben101Scf13863g00010.1 | VITSYAILETKLYNLGWERYYD--DPELLQYHKRC-TTHL-ISLPKDENKFKTM |
| SMEL_001g140830.1.01 | VITSYAVLERKLYSLGWERYYD--DPDLLQYHKRS-TVHL-ISLPKDENRFKSM |
| Capana01g003223 | VITSYAVLERKLYSLGWERYYD--EPELLQYHKRS-TVHL-ISLPKDENRFKSM |
| Solyc01g066980.3.1 | VITSYAVLERKLYSLGWERYYD--EPELLQYHKRS-TVHL-ISLPKDFNRFKSM |
| Sopen01g028640.1 | VITSYAVLERKLYSLGWERYYD--EPELLQYHKKS-TVHL-ISLPKDENRFKSM |
| Sopim01g066980.0.1 | VITSYAVLERKLYSLGWERYYD--EPELLQYHKRS-TVHL-ISLPKDENRFKSM |
| PGSC0003DMP400020089 | VITSYAVLERKLYSLGWERYYD--EPELLQYHKRS-TVHL-ISLPKDENRFKSM |
| Niben101Scf10524g05008.1 | VITSYPVLERKLYSLGWERYYD--DLNLLQYHKRS-TVHL-ISLPKDENKFKSM |
| Peaxi162Scf00534g00012.1 | VITSYALLERKLYSLGWERYYD--DPDLLQYHKRS-TVHL-ISLPKDENKEKSI |
| Peinf101Scf01113g00005.1 | VITSYALLERKLYSLGWERYYD--DPDLLQYHKRS-TVHL-ISLPKDENKEKSI |
| Peaxi162Scf00086g00036.1 | VITSYATLERKLYNLGWERYYD--DPELLQYHKRS-TVHL-ISLPKDFSRFKSM |
| Peinf101Scf00973g06042.1 | VITSYATLERKLYNLGWERYYD--DPELLQYHKRS-TVHL-ISLPKDFSRFKSM |
| Capana01g003222 | VISSYASLERKLYSLGWERYYD--DPDLLQYHKRS-TVHL-ISLPKDENRFKSM |
| Peaxi162Scf00534g00005.1 | VISSYATLERKLSSLGWERYYD--DPDLLQYHKRS-TVHL-ISLPKDENRFKSM |
| Peinf101Scf01113g00004.1 | VISSYATLERKLSSLGWERYYD--DPDLLQYHKRS-TVHL-ISLPKDENRFKSM |
| SMEL_001g140850.1.01 | VITSYAALERKLYSLGWERYYD--DPDLLQYHKRS-TVHL-ISLPKDENRFKSM |
| Niben101Scf02626g03001.1 | VITSYSALERKLYSLGWERYYD--DPDLLQYHKRS-TVHL-ISLPKDENKFKSM |
| Niben101Scf10524g05006.1 | VITSYSALERKLYSLGWERYYD--DPDLLQYHKRS-TVHL-ISLPKDENRFKSM |
| Solyc01g066970.2.1 | VITSYAVLERKLYSLGWERYYD--DPDLLQFHKRS-TVHL-ISLPKDENNLKSM |
| Sopen01g028630.1 | VITSYASLERILFSLGWERYYD--DPDLLQFHKRS-TIHL-ISLPKDENNFKSM |
| PGSC0003DMP400020088 | VITSYASLERNLYSLGWERYYD--DPDLLQFHKRS-TVHL-ISLPKDENRFKSM |
| Sopen01g028610.1 | VITSYASLERILYSLGWERYYD--DPNLLQYHKRS-TVHL-ISLPKDENNLKSM |
| Solyc01g066950.1.1 | VITSYAVLERKLYSLGWERYYD--DPDLLQYHKRS-TVHL-ISLPNDENNLRSM |
| Capana12g000135 | VITCYENLERKLCNLGWERFKSM |
| Capang12g000108 | VITCYENLERKLCNLGWERYYD--DPQLLQYHKRS-TIHL-ISLPLDFTRFKSM |
| SMEL 005g240480.1.01 | VITCYENLERKLYSLGWERYYD--DPOLLOYHKRS-TIHL-ISLPMDENRFKSM |
| Solyc12g099610.1.1 | VITCYANLEKKLYSLGWERYYD--DPQLLQYHKRS-TIHL-ISLPIDENRFKSI |
| PGSC0003DMP400008206 | VITCYANLERKLYSLGWERYYD--DPQLLQYHKRS-TIHL-ISLPIDENRFKSI |

(part E). Brachytic loci homologs, amino acid sequence alignment part 5.

| | |
|---|---|
| Niben101Scf00012g00011.1 | HIE |
| Peaxi162Scf00056g00139.1 | HMYDIVVKNRSFFEVRDSPYTSY |
| Peinf101Scf01105g01005.1 | HMYDIVVKNRSFFEVRDSPYTSY |
| Capana06g002723 | HMYDIVVKNPSFFEVRNAEVDNHLI |
| Capang06g002516 | HMYDIVVKNPSFFEVRNAEVDNHLI |
| Capang05g001509 | HMYDIVVKNPSFFEVRNAEVDNHLI |
| SMEL_006g247790.1.01 | QMYDIVVKNPSFFEVRDIKVYDHPI |
| PGSC0003DMP400007817 | QMYDIVVKNPSIFEVRDVKVCDHLI |
| Sopen06g001510.1 | NMYDIVVKNPSVFEVRDVKVCDHLI |
| Solyc06g005530.2.1 | NMYDIVVKNPSVFEVR |
| Niben101Scf05107g01003.1 | QMYDIVFKTRHIFHVRYI |
| Peinf101Scf02016g05027.1 | QEIVLKYCVGIKLSH |
| Peaxi162Scf00078g00059.1 | HMYDIVVKNPNVFHVRDA |
| SMEL_012g387130.1.01 | HMYDIVVKNPNIFHVRDV |
| Capana10g001758 | HMYDIVVKNPNVFHVRDV |
| CA05g11610 | HMYDIVVKNPNVFHVRDV |
| Niben101Scf05041g04001.1 | HMYDIVVKNPNVFHVRDA |
| Niben101Scf02182g12004.1 | HMYDIVVKNPNVFHVRDV |
| Sopen01g028590.1 | HMLDIVLKNRNDFTTRDTSITNNN |
| Niben101Scf13863g00010.1 | HMYDIVVKNRNEFEVRDM |
| SMEL_001g140830.1.01 | HMYDIVVKNRNEFEVREM |
| Capana01g003223 | HMFDIVVKNRNEFEVRDM |
| Solyc01g066980.3.1 | HMFDIVVKNRNEFEVRDM |
| Sopen01g028640.1 | HMFDIVVKNRNEFEVRDM |
| Sopim01g066980.0.1 | HMFDIVVKNRNEFEVRDM |
| PGSC0003DMP400020089 | HMFDIVVKNRNEFEVRDM |
| Niben101Scf10524g05008.1 | HMYDIVVKNRNEFEVRDT |
| Peaxi162Scf00534g00012.1 | HMYDIVVKNRNEFEVRDK |
| Peinf101Scf01113g00005.1 | QMYDIVVKNRNEFEVRDK |
| Peaxi162Scf00086g00036.1 | HMYDIVVKNRNEFEVRDM |
| Peinf101Scf00973g06042.1 | HMYDIVVKNRNEFEVRD |
| Capana01g003222 | HMYDIVVKNRNEFEVRDI |
| Peaxi162Scf00534g00005.1 | HMYDIVVKNRNEFEVRDM |
| Peinf101Scf01113g00004.1 | HMYDIVVKNRNEFEVRDM |
| SMEL_001g140850.1.01 | HMYDIVVKNRNEFEVRDM |
| Niben101Scf02626g03001.1 | HMYDIVVKNRNEFEVRDM |
| Niben101Scf10524g05006.1 | HMYDIVVKNRNEFEVRDM |
| Solyc01g066970.2.1 | HMYDIVVKNRNEFTVRDM |
| Sopen01g028630.1 | HMYDIVVKNRNEFTVRDM |
| PGSC0003DMP400020088 | HMYDIVVKNRNEFEVRDM |
| Sopen01g028610.1 | HMYDIVVKNRNEFTVRDM |
| Solyc01g066950.1.1 | HMYDIVVKNRNEFAVRDM |
| Capana12g000135 | HMYDIVVKNRNEFEVRDMWATRSTALRCEVQVMMDQPEVCADALDK |
| Capang12g000108 | HMYDIVVKNRNEFEVRDM |
| SMEL 005g240480.1.01 | HMYDIVVKNRNEFEVRDM |
| Solyc12g099610.1.1 | HMYDIVVKNRNEFEVRDM |
| PGSC0003DMP400008206 | HMYDIVVKNRNEFEVRDM |

(part F). Brachytic loci homologs, amino acid sequence alignment part 6.

| | | | |
|---|---|---|---|
| Niben101Scf00012g00011.1 | Nicotiana benthamiana | Tobacco | SEQ ID NO: 29 |
| Peaxi162Scf00056g00139.1 | Petunia axillaris | White Petunia | SEQ ID NO: 30 |
| Peinf101Scf01105g01005.1 | Petunia inflata | Petunia | SEQ ID NO: 31 |
| Capana06g002723 | Capsicum annuum Zunla | Pepper | SEQ ID NO: 32 |
| Capang06g002516 | Capsicum annuum Zunla | Pepper | SEQ ID NO: 33 |
| Capang05g001509 | Capsicum annuum | Pepper (Chiltepin) | SEQ ID NO: 34 |
| SMEL 006g247790.1.01 | Solanum melongena | Eggplant | SEQ ID NO: 35 |
| PGSC0003DMP400007817 | Solanum tuberosum | Potato | SEQ ID NO: 36 |
| Sopen06g001510.1 | Solanum pennellii | Wild tomato | SEQ ID NO: 37 |
| Solyc06g005530.2.1 | Solanum lycopersicum | Tomato | SEQ ID NO: 38 |
| Niben101Scf05107g01003.1 | Nicotiana benthamiana | Tobacco | SEQ ID NO: 39 |
| Peinf101Scf02016g05027.1 | Petunia inflata | Petunia | SEQ ID NO: 40 |
| Peaxi162Scf00078g00059.1 | Petunia axillaris | White Petunia | SEQ ID NO: 41 |
| SMEL_012g387130.1.01 | Solanum melongena | Eggplant | SEQ ID NO: 42 |
| Capana10g001758 | Capsicum annuum Zunla | Pepper | SEQ ID NO: 43 |
| CA05g11610 | Capsicum annuum | Pepper (CM334) | SEQ ID NO: 44 |
| Niben101Scf05041g04001.1 | Nicotiana benthamiana | Tobacco | SEQ ID NO: 45 |
| Niben101Scf02182g12004.1 | Nicotiana benthamiana | Tobacco | SEQ ID NO: 46 |
| Sopen01g028590.1 | Solanum pennellii | Wild tomato | SEQ ID NO: 47 |
| Niben101Scf13863g00010.1 | Nicotiana benthamiana | Tobacco | SEQ ID NO: 48 |
| SMEL_001g140830.1.01 | Solanum melongena | Eggplant | SEQ ID NO: 49 |
| Capana01g003223 | Capsicum annuum Zunla | Pepper | SEQ ID NO: 50 |
| Solyc01g066980.3.1 | Solanum lycopersicum | Tomato | SEQ ID NO: 51 |
| Sopen01g028640.1 | Solanum pennellii | Wild tomato | SEQ ID NO: 52 |
| Sopim01g066980.0.1 | Solanum pimpinellifolium | Wild tomato | SEQ ID NO: 53 |
| PGSC0003DMP400020089 | Solanum tuberosum | Potato | SEQ ID NO: 54 |
| Niben101Scf10524g05008.1 | Nicotiana benthamiana | Tobacco | SEQ ID NO: 55 |
| Peaxi162Scf00534g00012.1 | Petunia axillaris | White Petunia | SEQ ID NO: 56 |
| Peinf101Scf01113g00005.1 | Petunia inflata | Petunia | SEQ ID NO: 57 |
| Peaxi162Scf00086g00036.1 | Petunia axillaris | White Petunia | SEQ ID NO: 58 |
| Peinf101Scf00973g06042.1 | Petunia inflata | Petunia | SEQ ID NO: 59 |
| Capana01g003222 | Capsicum annuum Zunla | Pepper | SEQ ID NO: 60 |
| Peaxi162Scf00534g00005.1 | Petunia axillaris | White Petunia | SEQ ID NO: 61 |
| Peinf101Scf01113g00004.1 | Petunia inflata | Petunia | SEQ ID NO: 62 |
| SMEL_001g140850.1.01 | Solanum melongena | Eggplant | SEQ ID NO: 63 |
| Niben101Scf02626g03001.1 | Nicotiana benthamiana | Tobacco | SEQ ID NO: 64 |
| Niben101Scf10524g05006.1 | Nicotiana benthamiana | Tobacco | SEQ ID NO: 65 |
| Solyc01g066970.2.1 | Solanum lycopersicum | Tomato | SEQ ID NO: 66 |
| Sopen01g028630.1 | Solanum pennellii | Wild tomato | SEQ ID NO: 67 |
| PGSC0003DMP400020088 | Solanum tuberosum | Potato | SEQ ID NO: 68 |
| Sopen01g028610.1 | Solanum pennellii | Wild tomato | SEQ ID NO: 69 |
| Solyc01g066950.1.1 | Solanum lycopersicum | Tomato | SEQ ID NO: 70 |
| Capana12g000135 | Capsicum annuum Zunla | Pepper | SEQ ID NO: 71 |
| Capang12g000108 | Capsicum annuum | Pepper (Chiltepin) | SEQ ID NO: 72 |
| SMEL 005g240480.1.01 | Solanum melongena | Eggplant | SEQ ID NO: 73 |
| Solyc12g099610.1.1 | Solanum lycopersicum | Tomato | SEQ ID NO: 74 |
| PGSC0003DMP400008206 | Solanum tuberosum | Potato | SEQ ID NO: 75 |

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

The following examples are provided to illustrate certain particular features and/or embodiments. These examples should not be construed to limit the disclosure to the particular features or embodiments described.

### EXAMPLES

### Example 1

### Identification of Brachytic Homologs

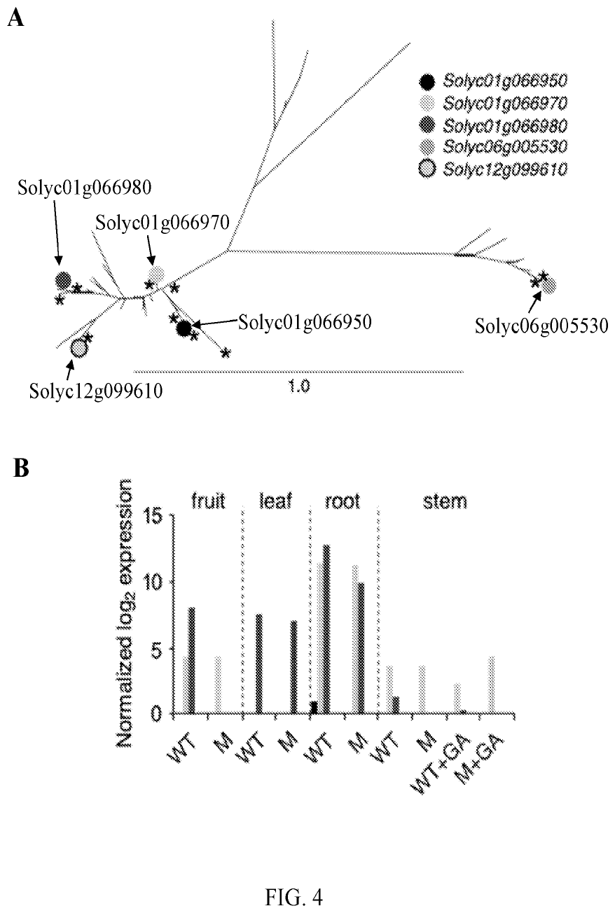

To identify the FPF (brachytic) gene family in Solanaceae, we performed a hidden Markov model (HMM) search using the PFAM FPF model against the 11 Solanaceae annotated protein datasets, including three tomato species, one modern (cultivated) (Solanum lycopersicum) and two wild tomatoes (S. pimpinellifolium and S. pennellii). We identified 57 protein sequences (including five modern tomato sequences) matching the model. For each of species, multiple sequences were identified in the datasets used in this study (ranging from three FPFs in Capsicum annuum cv. CM334 to eight in N. benthamiana). A maximum likelihood phylogenetic analysis revealed that five modern tomato sequences can be clustered into two categories (FIG. 4A). One contained all three FPFs on chromosome 1. The other category clustered all three tomato species, including a single modern tomato gene Solyc06g005530, close to a single terminal branch. Both wild tomatoes and modern tomato had five FPF1s. However, the modern tomato and its closest relative S. pimpinellifolium carried three FPFls on chromosome 1, while S. pennellii carried four FPF1s on chromosome 1, implying molecular divergence in the FPF1 family in Solanum.

To obtain an overview of the expression profiles of the five tomato FPF1s, RNA-seq libraries were constructed from different tissue types, the first internode (stem), leaf, and root at the 6-week-old growth stage (the growth stage used in conventional brachytic phenotyping; Lee et al., 2018). Additionally, first internodes collected 3 h after GA3 treatment at the 6-week-old stage were used for library construction. Comparing the expression profiles among homologs, both Br (Solyc01g066980) and its immediately adjacent gene Solyc01g066970 were expressed (FIG. 4B). Solyc01g066970 expression was not significantly affected by genotype. Notably, both genes were highly expressed in roots and expression levels of those two genes were not significantly affected by GA₃ treatment. The other three homologs had low expression levels in most or all tissue types.

RNAseq and expression analysis: Wild-type and mutant (M 2 generation of br.8.2^{CR}), tissue samples were collected from individual plants grown simultaneously with plants used to the greenhouse trial in the fall. Five different tissue types were collected: stem without GA₃ treatment (specifically the 1^{st} internode) at the 6-week-old stage, stem (specifically the 1^{st} internode) collected 3 h after GA₃ treatment at the 6-week-old stage, leaf at the 6-week-old stage, root at the 6-week-old stage, and fruit at the time of harvest. The leaf, stem with or without GA₃ treatment, and root samples were collected from 6-week-old plants. For each biological replication, the stem, leaf, and root were collected from the same individual plant, and four biological replications (four different plants) were collected for each genotype and tissue type. The samples were flash-frozen in liquid nitrogen immediately after excision.

### Example 2

### Gene Editing Tomato Plants Using CRISPR System

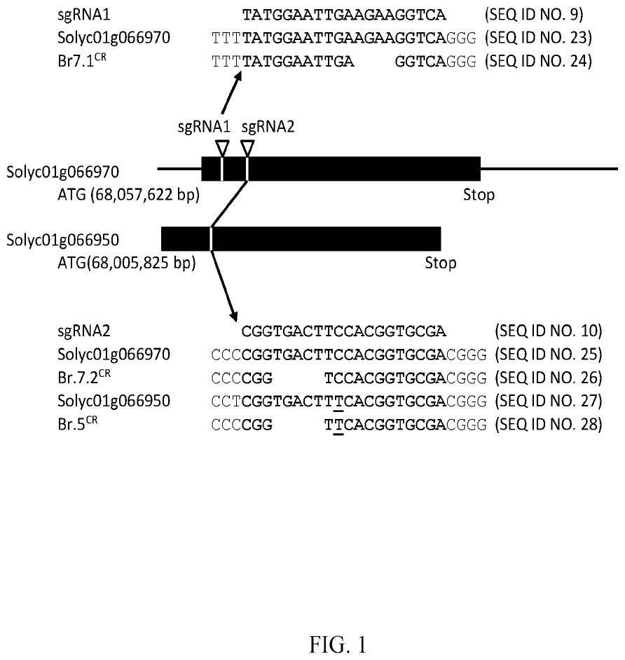

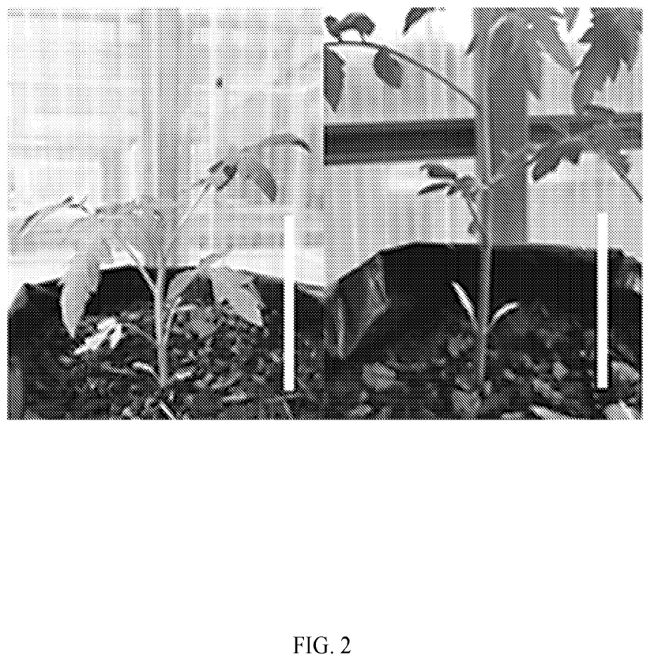

CRISPR constructs were designed to create deletions within the Solyc01g066970 and/or Solyc01g066950 loci the using sgRNA alongside the zCas9 endonuclease gene. zCas9 is a Cas9 gene that has been codon optimized for maize. Two different gRNA sequences containing SEQ ID NOs: 9 and 10 guide sequences were used to form CRISPR/zCas9 constructs to genetically modify the Solyc01g066970 and/or Solyc01g066950 loci in tomato plants to produce brachytic plants. The locations of the guide sequences relative to the Solyc01g066970 and Solyc01g066950 loci are illustrated in FIG. 1. All constructs were assembled as described by Xie et al. 2014 with minor modifications. pHSN401 vector (Addgene) was used to make the CRISPR/zCas9 constructs. Agrobacterium tumefaciens-mediated transformations of the standard fresh-market tomato (Solanum lycopersicum) variety Fla. 8059 were performed according to Van Eck et al. 2006 with minor modifications. Two different A. tumefaciens strains AGL1 (ATCC) and LBA4404 (Takara Bio USA), containing the indicted CRISPR/zCas9 constructs were used for transformations. After selecting regenerants on selecting media with hygromycin, regenerants were moved to the greenhouse. Young leaf tissues were collected from each TO plant, and genomic DNA was extracted using Qiagen DNeasy kit (Qiagen, USA). Each plant was genotyped for the presence of the CRISPR/zCas9 construct. Plants positive for Cas9 T-DNA were further genotyped for brachytic genome modification using Sanger.

The Solyc01g066970 locus and the Solyc01g066950 locus mutants were generated using the CRISPR/Cas9 system (Plant Physiology 2014 166:1292-1294). The gRNAs sequences used to target the locus are shown in FIG. 1. sgRNA1 targets the Solyc01g066970 locus. sgRNA2 targets both the Solyc01g066970 locus and the Solyc01g066950 locus. For the sgRNA, the tracrRNA component had the sequence: GTTTAGAGCTAGAAATAGCAAGTTAAAATA-AGGCTAGTCCGTTATCAACTTGAAAAAGTGGCACCGAGTCGGTGC (SEQ ID NO: 4) or an RNA equivalent thereof. The resulting constructs were introduced into Fla. 8059 (HORTSCIENCE 2008 43:2228-2230) background by Agrobacterium tumefaciens-mediated transformation.

As shown in FIG. 2, tomato plants having CRISPR/zCas9-induced deletions in the Solyc01g066970 and Solyc01g066950 loci exhibited the brachytic phenotype, shortened height and decreased internode length (compare left (genetically modified) plants and right (normal) plants and in FIG. 2. The genetically modified plants contained 4 and 5 base pair deletions in the Solyc01g066970 locus and a 5 base pair deletion in the Solyc01g066950 locus (FIG. 1).

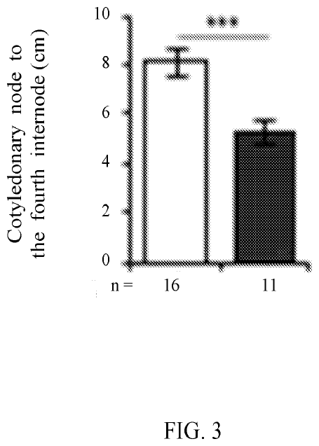

As illustrated in FIG. 3, the double mutant plants (white bar) had statistically reduced internode length. Shortened internode length was also observed in Solyc01g066970-mutant plants generated using a single sgRNA, sgRNA1.

### Example 3

### Mutated br Homologs Present New Sources of a Reduced Plant Height

Considering the observed sequence variation and expression patterns of FPFs adjacent to the Br (Solyc01g066980) on chromosome 1, we investigated phenotypes associated with mutated versions of those two br homologs, Solyc01g066950 and Solyc01g066970.

Guide RNAs (gRNAs) targeting FPF (Br) genes were designed using CRISPR-P (Lei et al., 2014) and CRISPR-PLANT (Xie et al., 2014) and each of the gRNAs was cloned into a binary vector following the same basic procedures described by Xie and Yang (2013) (Table 3). Duplex oligos carrying BsaI sites in binary vectors were synthesized (IDT). The binary vector pHSN401 (www.addgene.org)-gRNA plasmid was introduced into Agrobacterium tumefaciens strain LBA4404 (Takara, www.takarabio.com) according to the manufacturer's instructions. A. tumefaciens-mediated transformations of Fla. 8059 [A parental line of ‘Tasti-Lee Fi’ (Bejo, Seeds, Oceano, CA), Scott et al., 2008; Tasti-Lee Fi is a fresh-market tomato cultivar currently in the US market (e.g., Publix Super Markets, Inc., www.publix.com)] were performed as described by Van Eck et al., 2019, with modifications in the preculture medium and selective regeneration medium steps: Cotyledon explants from 7 to 9-day-old seedlings were precultured and 3 mg/L or 6 mg/L hygromycin was used.

Potential Cas9-gRNA-introduced mutations were examined by Sanger sequencing of PCR products and the T7 Endonuclease I assay (NEB) using the PCR primers in Table 4. Total genomic DNA of each transformed plant in the Mo generation was extracted from young leaves using the DNeasy Plant Mini Kit (Qiagen, www.qiagen.com). PCRs were performed to examine mutations in the targeted region. PCR cycling and running parameters were as follows: initial denaturation step at 95° C. for 7 min, 30 cycles at 95° C. for 30 s, 60° C. for 30 s, and 72° C. for 1 min, followed by a final extension at 72° C. for 7 min. For the T7 Endonuclease I assay, genomic DNA extracted from individual plants was used as the template. A pair of targeted region-specific primers and Q5 Hot Start High-Fidelity 2× Master Mix (NEB) were used for PCR. The cycling and running parameters were as follows: initial denaturation step at 98° C. for 30 s, 35 cycles at 98° C. for 5 s, 60° C. for 10 s, and 72° C. for 20 s, followed by a final extension at 72° C. for 2 min. PCR products were purified using a QIAquick PCR Purification Kit (Qiagen), and 200 ng of the PCR products was digested with T7E1 according to the manufacturer's instructions. To identify homozygous transgene-free mutants, four primer pairs targeting the Cas9 gene in the binary vector or the Hyg gene were used. Potential transgene-free mutants were further validated by whole genome sequencing. Potential off-target sites (i.e., up to four mismatches compared to each target region) were predicted using the Cas-OFFinder (Bae et al., 2014). A lack of off-target activity was verified (Table 5).

TABLE 3
guide RNAs

| Oligo | Sequence | SEQ ID NO. | Target |
|---|---|---|---|
| sgRNA1 | ATCGGAGTTC | 115 | Solyc01g066980 |
| | TCCACTAGA | | |
| sgRNA2 | GAAGATGTAC | 116 | Solyc01g066980 |
| | AAGAACTTTT | | |
| sgRNA3 | TCGCACCGTGA | 117 | Solyc01g066950 |
| | AAGTCACCG | | & |
| | | | Solyc01g066970 |

TABLE 4
PCR primers for mutation detection

| | | SEQ ID | |
|---|---|---|---|
| Oligo | Sequence | NO. | Target |
| Br_80_F | TTCCCCTCTT | 118 | Solyc01g066980 |
| | ACAACTTTCC | | |
| | AA | | |
| Br_80_R | CCAGAAACGG | 119 | |
| | GGGAGACTAC | | |
| Br_70_F | CATGTGCATG | 120 | Solyc01g066970 |
| | GACTTGAGGT | | |
| | TG | | |
| Br_70_R | AGGGCTGATC | 121 | |
| | AAGCAATGGA | | |
| | T | | |
| Br_50_F | GACCTGAGGT | 122 | Solyc01g066950 |
| | TGTTGAAGTC | | |
| | GT | | |
| Br_50_R | TTTTGGGTCG | 123 | |
| | TGACAGGTAA | | |
| | A | | |
| Cas9_F11 | CCAGATTCAT | 124 | Cas9 |
| | CTCGGGGAGC | | |
| Cas9_R11 | GAGCTGCTTA | 125 | |
| | ACCGTGACCT | | |
| Cas9_F12 | GGACTTCCTG | 126 | Cas9 |
| | GACAACGAGG | | |
| Cas9_R12 | CGTGAGTTCT | 127 | |
| | TCTGGCCCTT | | |
| Hyg_F2 | GAGGGCGTGG | 128 | HygR |
| | ATATGTCCTG | | |
| Hyg_R2 | GGCGACCTCG | 129 | |
| | TATTGGGAAT | | |
| Hyg_F11 | GCTCTCGATG | 130 | HygR |
| | AGCTGATGCT | | |
| Hyg_R11 | ATTTGTGTAC | 131 | |
| | GCCCGACAGT | | |

TABLE 5
Potential off-targets

| guide | | SEQ ID | | Position | | |
|---|---|---|---|---|---|---|
| RNAª | Potential off-target ^{b} | NO. | Chrom.^{c} | (bp) ^{d} | Strand ^{e} | Mismatches |
| sgRNA1 | GAaCGtAGTTgaCCACTAGATGG | 132 | 7 | 13,262,523 | minus | 4 |
| | GATtGaAGTTCTCCgtTAGATGG | 133 | 8 | 14,774,353 | minus | 4 |
| | cAatGGAGTTCTtCACTAGAGGG | 134 | 10 | 26,886,339 | minus | 4 |
| | GATgaGAGTTCTgCACTtGATGG | 135 | 11 | 46,729,949 | minus | 4 |
| sgRNA2 | ttAtGATGTACAAaAACTTTTAGG | 136 | 1 | 2,916,807 | plus | 4 |
| | GGAAGATGTACcAatACgTTTCGG | 137 | 1 | 27,276,750 | plus | 4 |
| | ttAAGATtTACAACAACTTTTTGG | 138 | 1 | 78,063,314 | minus | 4 |
| | GGAAGATGTcCtAGttCTTTTTGG | 139 | 1 | 81,784,871 | minus | 4 |
| | GGAAcATGTACAAGAAgcTTgAGG | 140 | 1 | 85,341,524 | minus | 4 |
| | GGAAGAcGTtCAAGAAtTTTTCGG | 141 | 2 | 22,562,061 | plus | 3 |
| | GGAAGATGaAtAAtAACTaTTTGG | 142 | 3 | 27,226,946 | plus | 4 |
| | aacAGAaGTACAAGAACTTTTGGG | 143 | 5 | 16,839,941 | plus | 4 |
| | aGcAGATGTACAAGAtCTTTaAGG | 144 | 5 | 46,179,653 | minus | 4 |
| | aGAAGcTGTAtAtGAACTTTTGGG | 145 | 6 | 46,988,801 | plus | 4 |
| | GGAAGAaGaAgAAGAAgTTTTAGG | 146 | 7 | 7,766,201 | plus | 4 |
| | tGAtGATGTAaAAGAACTTTTTGG | 147 | 7 | 44,564,055 | minus | 3 |
| | GGAAGATGgACAAcAAgaTTTAGG | 148 | 8 | 21,797,045 | plus | 4 |
| | tGAAGAaGcACAAGAgCTTTTTGG | 149 | 8 | 36,797,374 | minus | 4 |
| | GGAtGATaTACAAGcAtTTTTAGG | 150 | 8 | 56,549,095 | minus | 4 |
| | GGAAGATGTACcAtAACTTTaGGG | 151 | 9 | 41,907,371 | minus | 3 |
| | GcAAGATGcACAAGAcCcTTTGGG | 152 | 9 | 48,273,212 | plus | 4 |
| | GcAAGATcTACAAGAACTTcaCGG | 153 | 10 | 1,415,058 | plus | 4 |
| | GGAAGATaTtCAAtAAaTTTTAGG | 154 | 11 | 53,105,006 | minus | 4 |
| | GGAAGATaTgaAAGAACTTTaTGG | 155 | 12 | 29,596,866 | minus | 4 |

^{a} No potential off-targets were found for the sgRNA3 in this study.
^{b} Potential off-targets with a maximum mismatch of four were identified. Small letters indicate mismatches compared to each target region.
^{c}Chromosome, tomato reference genome assembly SL4.0.
^{d} position relative to the first nucleotide of each target region.
e DNA strand orientation

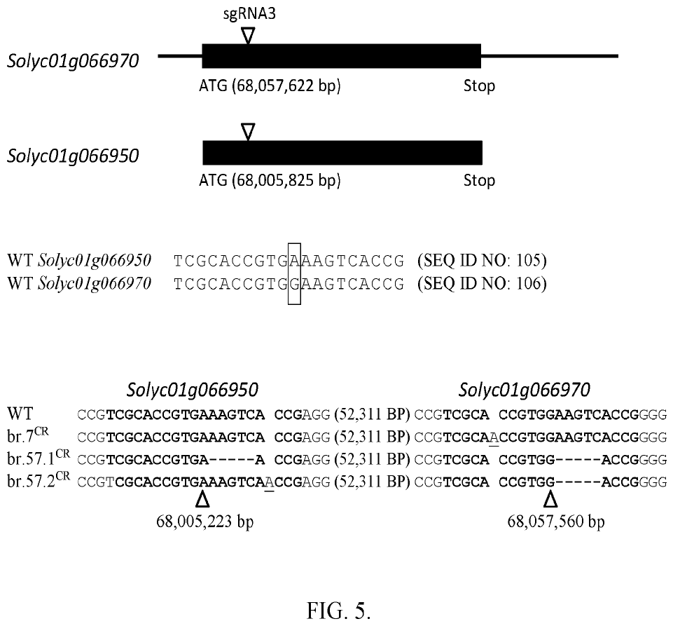

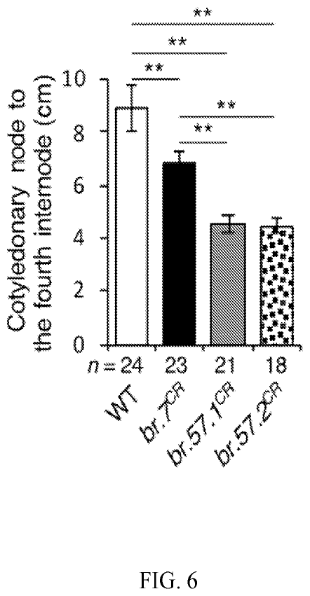

Using a single-guide RNA targeting a sequence region only differentiated by a single nucleotide, three different mutants were obtained simultaneously (FIG. 5): br.7^{CR}, having a 1 bp insertion in Solyc01g066970; br.57.1 cR , having a 5 bp deletion in both Solyc01g066950 and Solyc01g066970; and br.57.2^{CR}, having a 1 bp insertion in Solyc01g066950 and a 5 bp deletion in Solyc01g066970. None of these mutants had DNA sequence variation in Br (Solyc01g066980). All three mutants showed significantly reduced height (FIG. 6). As the number of genes knocked out increased, the stem length reduced accordingly. The findings indicate that multiple br homologs confer a br plant-like shortened stem length.

The data demonstrate that CRISPR-mediated knock-out(s) of Br homologs can confer a br plant-like shortened architecture (reduced plant height), while retaining the production of heavy fruits.

High levels of genetic variation [e.g., copy number variation of DNA segments (CNV)], have been observed in plant genomes, and emerging evidence indicates that CNVs mediate a number of valuable crop traits [for example, CNV (1 to 11 copies)-mediated soybean cyst nematode resistance]. Together with these results, this suggests creation of tomato lines that carry mutations in multiple FPF1 genes (e.g., knock-outs of 2, 3, 4, or all 5 of the br homologs) will be useful in generating tomato plants having a brachytic phenotype and large (medium or larger) fruit. CRISPR mediated knockout of two or more Br homolog genes may result in considerably reduced plant architectures than those obtained by single mutants.

### Example 4

### Generation of Loss of Function Mutations at Other Brachytic Loci Using CRISPR Systems

Identification of protospacer-adjacent motif (PAM) sites in the, Solyc01g066950, Solyc01g066970, Solyc06g005530, Solyc12g099610, and Solyc01g066980 genes for CRISPR/zCas9 generation of brachytic plants. In addition to the guide sequences described above, additional guide sequences are suitable for forming gRNAs (as used herein gRNA can include crRNA, gRNA, and sgRNA) for CRISPR/zCas9 mediated genetic modification of a br locus. Suitable guide sequences include 17-20 nucleotide sequences in SEQ ID NOs: 1, 2, 6, 7, 11, 12, 16, 17, 21, or 102 or a complement thereof that are unique compared to the rest of the genome and immediately adjacent (5′) to a protospacer-adjacent motif (PAM) site. For zCas9, a PAM site is NGG. Thus, any unique 17-20 nucleotide sequence immediately 5′ of a 5′-NGG-3′ in in SEQ ID NOs: 1, 2, 6, 7, 11, 12, 16, 17, 21, or 102 or a complement thereof can be used in forming a gRNA. PAM sites in the SEQ ID NOs: 1, 2, 6, 7, 11, 12, 16, 17, 21, and 102 are shown in Table 1, where GG and CC PAM sites are shown in capital letters. CC sequences in the listed strand correspond to GG sequences in the complement strand. Deletions or insertions in the flanking regions may alter expression of the brachytic gene leading to plants displaying a brachytic phenotype.

CRISPR modification of the brachytic locus is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

In some embodiments, two or more gRNAs can be used. The two or more gRNAs can used with the same RNA-guided DNA endonuclease (Cas nuclease) or different RNA-guided DNA endonucleases. CRISPR mediated modification of other brachytic loci, such as the Solyc06g005530 locus or the Solyc12g099610 locus, in tomato plants is accomplished in a similar manner by selecting target sequences as described in example 3 for Solyc01g066950 and Solyc01g066970.

CRISPR mediated modification of homologous or orthologous brachytic loci in other Solanaceae plants is accomplished in a similar manner by selecting target sequences as described in example 3 for Solyc01g066950 and Solyc01g066970. Exemplary homologous brachytic amino acid sequences are provided in Table 2.

1. A genetically modified Solanaceae plant wherein one or more of a Solyc01g066950 locus, a Solyc01g066970, a Solyc06g005530 locus, and a Solyc12g099610 locus has been genetically modified through the use of a CRISPR/Cas system.

2. The genetically modified Solanaceae plant of claim 1, wherein the Solanaceae plant has been genetically modified through the use of a CRISPR/Cas system at:
(a) a Solyc01g066950 locus and a Solyc01g066970 locus;
(b) a Solyc01g066950 locus and a Solyc06g005530 locus;
(c) a Solyc01g066950 locus and a Solyc12g099610 locus;
(d) a Solyc01g066950 locus and a Solyc01g066980 locus;
(e) a Solyc01g066970 locus and Solyc06g005530 locus;
(f) a Solyc01g066970 locus and Solyc12g099610 locus;
(g) a Solyc01g066970 locus and Solyc01g066980 locus;
(h) a Solyc06g005530 locus and Solyc12g099610 locus;
(i) a Solyc06g005530 locus, and Solyc01g066980 locus; or
(j) a Solyc12g099610 locus, and Solyc01g066980 locus;

3. The genetically modified Solanaceae plant of claim 1, wherein the Solanaceae plant has been genetically modified through the use of a CRISPR/Cas system at:
(a) a Solyc01g066950 locus, a Solyc01g066970 locus, and a Solyc06g005530 locus;
(b) a Solyc01g066950 locus, a Solyc01g066970 locus, and a Solyc01g066980 locus;
(c) a Solyc01g066950 locus, a Solyc01g066970 locus, and a Solyc12g099610 locus;
(d) a Solyc01g066950 locus, a Solyc06g005530 locus, and a Solyc12g099610 locus;
(e) a Solyc01g066950 locus, a Solyc06g005530 locus, and a Solyc01g066980 locus;
(f) a Solyc01g066950 locus, a Solyc12g099610 locus, and a Solyc01g066980 locus;
(g) a Solyc01g066970 locus, a Solyc06g005530 locus, and a Solyc12g099610 locus;
(h) a Solyc01g066970 locus, a Solyc06g005530 locus, and a Solyc01g066980 locus;
(i) a Solyc01g066970 locus, a Solyc12g099610 locus, and a Solyc01g066980 locus;
or
(j) a Solyc06g005530 locus, a Solyc12g099610 locus, and a Solyc01g066980 locus.

4. The genetically modified Solanaceae plant of claim 1, wherein the Solanaceae plant has been genetically modified through the use of a CRISPR/Cas system at:
(a) a Solyc01g066950 locus, a Solyc01g066970 locus, a Solyc06g005530 locus, and a Solyc12g099610 locus;
(b) a Solyc01g066950 locus, a Solyc01g066970 locus, a Solyc06g005530 locus, and a Solyc01g066980 locus;
(c) a Solyc01g066950 locus, a Solyc01g066970 locus, a Solyc12g099610 locus, and a Solyc01g066980 locus;
(d) a Solyc01g066950 locus, a Solyc06g005530 locus, a Solyc12g099610 locus, and a Solyc01g066980 locus; or
(e) a Solyc01g066970 locus, a Solyc06g005530 locus, a Solyc12g099610 locus, and a Solyc01g066980 locus,

5. The genetically modified Solanaceae plant of claim 1, wherein the Solanaceae plant has been genetically modified through the use of a CRISPR/Cas system at: a Solyc01g066950 locus, a Solyc01g066970 locus, a Solyc06g005530 locus, a Solyc12g099610 locus, and a Solyc01g066980 locus.

6. The genetically modified Solanaceae plant of any one of claims 1-5, wherein the genetically modified plant contains a deletion one or more of: the Solyc01g066950 locus, the Solyc01g066970 locus, the Solyc06g005530 locus, and the Solyc12g099610 locus.

7. A method of genetically modifying a Solyc01g066950 locus and/or a Solyc01g066970 locus in a Solanaceae plant, the method comprising: introducing a CRISPR system into a Solanaceae plant cell, wherein the CRISPR system comprises (a) an RNA-guided DNA endonuclease or a nucleic acid encoding the RNA-guided DNA endonuclease and (b) a guide RNA or a nucleic acid encoding the guide RNA into a plant cell; wherein the RNA-guided DNA endonuclease and the guide RNA protein form a complex that targets the Solyc01g066950 (SEQ ID NO: 1) locus and/or the Solyc01g066970 locus (SEQ ID NO: 6).

8. The method of claim 7, wherein genetically modifying the Solyc01g066950 locus and/or the Solyc01g066970 locus comprises generating a disruption of the Solyc01g066950 locus and/or the Solyc01g066970 locus.

9. The method of claim 7, wherein the CRISPR system is selected from the group consisting of: a CRISPR class 1 system, a CRISPR class 2 system, a CRISPR/Cas system, a CRISPR/Cas9 system, a CRISPR/zCas9 system and a CRISPR/Cas3 system.

10. The method of claim 7, wherein the RNA-guided DNA endonuclease comprises a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

11. The method of claim 7, wherein the guide RNA comprises a CRISPR RNA (crRNA) and a trans-activating CRISPR RNA (tracrRNA) as separate molecules or as a single chimeric guide RNA (sgRNA).

12. The method of claim 7, wherein introducing a CRISPR system into a Solanaceae plant cell comprises electroporation, microprojectile bombardment, biolistic transformation, microinjection, protoplast transformation, an Agrobacterium tumefaciens vector transformation or an Agrobacterium rhizogenes vector transformation.

13. The method of claim 7, wherein the guide RNA comprises:
(a) a 17-20 nucleotide guide sequence comprising 17-20 contiguous nucleotides differing by no more than 1 or 2 nucleotides present in SEQ ID NO: 1 or a complement thereof or an ortholog thereof, and/or
(b) a 17-20 nucleotide guide sequence comprising 17-20 contiguous nucleotides differing by no more than 1 or 2 nucleotides present in SEQ ID NO: 6 or a complement thereof or an ortholog thereof;
wherein the 17-20 nucleotide sequence is unique compared to the rest of the genome of the Solanaceae plant and is immediately adjacent (5′) to a protospacer-adjacent motif (PAM) site.

14. The method of claim 13, wherein the guide RNA contains comprises:
(a) a 17-20 nucleotide guide sequence comprising 17-20 contiguous nucleotides differing by no more than 1 or 2 nucleotides present in SEQ ID NO: 2 or a complement thereof or an ortholog thereof, or
(b) a 17-20 nucleotide guide sequence comprising 17-20 contiguous nucleotides differing by no more than 1 or 2 nucleotides present in SEQ ID NO: 7 or a complement thereof or an ortholog thereof.

15. The method of claim 13, wherein the PAM site is selected from the group consisting of: 5′-NGG-3′, 5′-NNNNGATT-3′, 5′-NNAGAA-3′, and 5i-NAAAAC-3′.

16. The method of claim 13, wherein the guide RNA comprises a nucleic acid sequence selected from the group consisting of: SEQ ID NO: 5, SEQ ID NO: 9, SEQ ID NO: 10, or SEQ ID NO: 117, or an RNA equivalent thereof.

17. The method of claim 7, wherein the CRISPR system further comprises a second guide RNA.

18. The method of claim 17, wherein CRISPR system comprises a single RNA-guided DNA endonuclease or two different RNA-guided DNA endonucleases.

19. The method of claim 17, wherein the guide RNA comprises SEQ ID NO: 9 or an RNA equivalent thereof and the second guide RNA contains the sequence of SEQ ID NO: 10 or an RNA equivalent thereof.

20. The method of claim 7, wherein the CRISPR system creates a deletion of one or more nucleotides in the Solyc01g066950 locus and/or the Solyc01g066970 locus.

21. The method of claim 20, wherein the deletion comprises a 1-5 base pair deletion.

22. The method of claim 7, wherein the Solanaceae plant comprises a tomato plant.

23. The method of claim 7, wherein the method comprises generating one or more regenerants following introducing the CRISPR system into a Solanaceae plant cell.

24. The method of claim 7, wherein the method further comprises genotyping one or more regenerants for the presence of a the Solyc01g066950 locus modification and/or a Solyc01g066970 locus modification.

25. The method of claim 24, wherein the method further comprises selecting one or more To plants containing a genomic modification at the Solyc11 g066950 locus and/or the Solyc01g066970 locus.

26. The method of claim 7, wherein genetically modifying the Solyc01g066950 locus and/or the Solyc01g066970 locus in a Solanaceae plant results in the Solanaceae plant having shortened height and/or decreased internode length.

27. A method of genetically modifying a Solanaceae plant to produce a plant having a brachytic phenotype, the method comprising: introducing a Cas protein or a nucleic acid encoding the Cas protein and a guide RNA or a nucleic acid encoding the guide RNA into a plant cell, wherein the guide RNA and Cas protein form a complex that targets a target sequence in one or more of: SEQ ID NO: 1, SEQ ID NO: 2, SEQ ID NO: 6, SEQ ID NO: 7, SEQ ID NO: 11, SEQ ID NO: 12, SEQ ID NO: 16, and SEQ ID NO: 17.

28. The method of claim 27, further comprising introducing a second guide RNA or a nucleic acid encoding the second guide RNA into a plant cell, wherein the second guide RNA forms a complex with the Cas protein that targets a target sequence in SEQ ID NO: 21 or 102.

29. A method of genetically modifying a Solyc06g005530 locus and/or a Solyc12g099610 locus in a Solanaceae plant, the method comprising: introducing a CRISPR system into a Solanaceae plant cell, wherein the CRISPR system comprises an RNA-guided DNA endonuclease or a nucleic acid encoding the RNA-guided DNA endonuclease and a guide RNA or a nucleic acid encoding the guide RNA into a plant cell, wherein the RNA-guided DNA endonuclease and the guide RNA protein form a complex that targets the Solyc06g005530 locus and/or the Soly12g099610 locus.

30. The method of claim 32, wherein the guide RNA comprises a nucleic acid sequence selected from the group consisting of: SEQ ID NOs: 14-15, 19-20, 76-92, and 93-101.

31. A method of genetically modifying a tomato plant, the method comprising: introducing a CRISPR system into a tomato plant cell, wherein the CRISPR system comprises an RNA-guided DNA endonuclease or a nucleic acid encoding the RNA-guided DNA endonuclease and a guide RNA or a nucleic acid encoding the guide RNA into a plant cell, wherein the RNA-guided DNA endonuclease and the guide RNA protein form a complex that targets one or more of a Solyc01g066950 locus, a Solyc01g066970 locus, a Solyc06g005530 locus, and a Solyc12g099610 locus.

32. A method of generating a Solanaceae plant having a brachytic phenotype comprising: introducing a CRISPR system into a Solanaceae plant cell, wherein the CRISPR system comprises an RNA-guided DNA endonuclease or a nucleic acid encoding the RNA-guided DNA endonuclease and a guide RNA or a nucleic acid encoding the guide RNA into a plant cell, wherein the RNA-guided DNA endonuclease and the guide RNA protein form a complex that targets the Solyc01g066950 locus, the Solyc01g066970 locus, the Solyc06g005530 locus, and/or the Solyc12g099610 locus thereby generating a loss of function mutation at the Solyc01g066950 locus, the Solyc01g066970 locus, the Solyc06g005530 locus, and/or the Solyc12g099610 locus, and generating a regenerant plant from the Solanaceae plant cell.

33. The method of claim 32, further comprising introducing a second guide RNA or a nucleic acid encoding the second guide RNA into a plant cell, wherein the second guide RNA forms a complex with the Cas protein that targets a target sequence in SEQ ID NO: 21 or 102.
